(19) United States
(12) Patent Application Publication
(10) Pub. No.: US 2020/0051666 A1
(43) Pub. Date: Feb. 13, 2020

(54) HLA-INDEXED REPOSITORY OF iPSCS AND iPSC-DERIVED STEM CELLS, AND RELATED SYSTEMS AND METHODS

(71) Applicant: Orig3n, Inc., Boston, MA (US)

(72) Inventors: Robin Y. Smith, Boston, MA (US); Marcie A. Glicksman, Boston, MA (US)

(21) Appl. No.: 16/492,690

(22) PCT Filed: Mar. 14, 2018
(86) PCT No.: PCT/US18/22317

§ 371 Date: Sep. 10, 2019

Related U.S. Application Data
(60) Provisional application No. 62/517,600, filed on Jun. 9, 2017.
(60) Provisional application No. 62/513,966, filed on Jun. 1, 2017.
(60) Provisional application No. 62/513,032, filed on May 31, 2017.
(60) Provisional application No. 62/513,380, filed on May 31, 2017.
(60) Provisional application No. 62/471,310, filed on Mar. 14, 2017.

(51) Int. Cl. G16B 35/20; A61K 35/28; A61K 35/545; G06F 16/22; C12N 5/074; C40B 40/02; G16H 50/70; G16H 50/20

(52) U.S. Cl. CPC G16B 35/20; A61K 35/28; A61K 35/545; G16H 50/20; C12N 5/0696; C40B 40/02; G16H 50/70; G06F 16/22

(57) ABSTRACT

Described herein is a managed, indexed repository of hematopoietic stem cell (HSC) lines and/or blood progenitor cell lines derived from induced pluripotent stem cells (iPSCs) (or embryoid bodies formed from iPSCs), wherein each of the HSC lines, blood progenitor cell lines, embryoid bodies, and/or iPSC lines has corresponding data comprising a set of characterized HLA loci, said corresponding data being stored in a searchable database for retrieval of one or more matching physical cell lines upon query. The repository comprises an HLA-indexed bank of iPSCs, embryoid bodies, HSCs, and/or blood progenitor cells for each of a set of HLA types, for identification and provision of allogeneic cell lines suitable for transplantation to reestablish hematopoietic function in patients with damaged, diseased, or otherwise abnormal bone marrow and/or immune system.

### CROSS REFERENCE TO RELATED APPLICATIONS

This application claims the benefit of U.S. Provisional Application No. 62/471,310 filed Mar. 14, 2017, U.S. Provisional Application No. 62/513,032 filed May 31, 2017, U.S. Provisional Application No. 62/513,380 filed May 31, 2017, U.S. Provisional Application No. 62/513,966 filed Jun. 1, 2017, and U.S. Provisional Application No. 62/517,600 filed Jun. 9, 2017, the contents of which are hereby incorporated by reference herein in their entirety.

### FIELD OF THE INVENTION

The invention relates generally to repositories of characterized induced pluripotent stem cells (iPSCs), cells derived therefrom, and/or associated cell lines. Further, in certain embodiments, the invention relates generally to apparatus, systems, and methods for identification of one or more human leukocyte antigen (HLA) loci-matched physical cells and/or cell lines from a repository of iPSCs and/or cells derived therefrom.

### BACKGROUND

Human cells have two sets of markers, one inherited from each parent, that communicate to immune cells, instructing them to ignore one's own cells rather than attack them. The chance of transplant rejection is reduced with a perfect or close match to these markers. Unfortunately, finding a good cell or tissue match can take years, as witnessed by the long waiting periods on organ donation lists.

Autologous transplantation of iPSCs has attempted to reduce the rate of rejected organ transplants. In autologous transplantation, cells have to be prepared for every individual, thereby making the process very expensive and time consuming (e.g., from about 6 to 9 or more months). On the other hand, allogeneic iPSCs can be prepared for large groups of individuals. Allogeneic cells are made in advance and can be ready when people need them. Fewer allogeneic lines are needed to serve a population. Moreover, iPS cells function like embryonic stem cells in that they can be differentiated into a variety of different cell types.

The term “super donors” refers to human leukocyte antigen (HLA) types (or cell lines or individuals having those HLA types) that do not trigger strong rejection reactions. Super donors have a HLA haplotype that is common among the population and will match a sizable portion of a particular population. This is analogous to banking a blood transfusion from a donor who has blood type O-negative, which can be tolerated by patients of all blood types.

Humans are almost always heterozygous for a particular HLA gene—that is, humans usually express two different alleles. For successful transplantation, eight (8) HLA alleles are best for matching (4 alleles on each of the donor and recipients chromosomes). With homozygous donors, only 4 alleles are required to be matched, therefore increasing the number of recipients that would be a match to the donor. Individuals that are homozygous for all three key HLA alleles that govern rejection means that only three genes need to be matched instead of six genes. Accordingly, iPSC lines derived from these so-called “super donors” can be used to reduce immunogenicity. It is believed that about 200 such iPSC lines could cover a high percentage (e.g., at least 90%, at least 95%, or more) of the U.S. and/or European population, and about 90 to 100 such iPSC lines could cover a high percentage (e.g., at least 90%, at least 95%, or more) of the Japanese population.

Bone marrow transplantation generally involves replacing damaged bone marrow with healthy bone marrow stem cells. Hematopoietic stem cells (HSCs) can be used in transplantation and can be derived from bone marrow, peripheral blood, or umbilical cord blood. Where an allogeneic transplant is needed, a suitable donor (someone other than the patient) must be found for the patient in order to minimize risk of transplant rejection and maximize chances for success. Bone marrow donor registries are services that seek to match registered donors with patients in need of an allogeneic transplant. Matching based on human leukocyte antigen (HLA) typing is typically performed to find suitable donors. Because there are many different HLA types, it is often difficult to find suitable matches, particularly when no family members of the patient are an HLA-identical match.

There is a need for alternatives to existing donor registry systems for matching donors to patients.

### SUMMARY

Described herein is an alternative to existing inefficient donor registry systems for matching donors to patients. For example, a repository of iPSCs has been assembled with indexed HLA haplotypes for a given population that matches to a large percentage of the population. Also described herein are methods for identifying indexed HLA iPSCs, e.g., suitable for iPSC-derived HSC transplantation. In certain embodiments, reserves of iPSCs and other iPSC-derived cells (e.g., hematopoietic stem cell (HSCs), blood progenitor cells, Retinal Pigment Epithelium (RPE), chondrocytes, mesenchymal stem cells (MSCs), and embryoid bodies), iPSC lines and other iPSC-derived cell lines (e.g., HSC lines, blood progenitor cell lines, RPE lines, MSC lines, and embryoid bodies) are stored in a managed physical repository (e.g., a bank) for providing a resource (e.g., donors for organ transplants) for patients. This managed repository of HSC lines and/or blood progenitor cell lines derived from induced pluripotent stem cells (iPSCs) (or embryoid bodies formed from iPSCs), also stores corresponding characterization data comprising a set of characterized HLA loci, said corresponding characterization data being stored in a searchable database for retrieval of one or more matching physical cells and/or cell lines upon query. The repository comprises a bank of HLA-indexed cells (e.g., iPSCs, embryoid bodies, HSCs, MSCs, RPEs, blood progenitor cells, chondrocyte, and/or various other cells) and/or cell lines (e.g., iPSC lines, HSC lines, MSC lines, RPE lines, blood progenitor cell lines and/or various other cell lines derived from iPSCs). In certain embodiments, the cells and cell lines comprise iPSCs, embryoid bodies, HSCs, blood progenitor cells, MSCs, RPEs, chondrocytes, and/or various other cells and/or cell lines derived from iPSCs for each of a set of HLA types. In certain embodiments, the HLA-indexed cells and/or cell lines may be identified through the searchable database and may be used for provision of allogeneic cell lines suitable for transplantation to reestablish hematopoietic function in patients with damaged, diseased, or otherwise abnormal bone marrow and/or immune system.

Further, described herein are systems and methods for identification of matching human leukocyte antigen (HLA) loci from HLA-indexed repositories. More particularly, in certain embodiments, the invention relates to a repository of characterized (e.g., HLA-indexed, ABO-indexed, RHD indexed, and the like) induced pluripotent stem cells (iPSCs) and/or iPS cell lines and/or cells derived therefrom, wherein each of the cell lines has corresponding data comprising a set of characterized (e.g., mapped and indexed) HLA loci, said corresponding data being stored in a searchable database for retrieval of one or more matching physical cells and/or cell lines upon query. In certain embodiments, the iPS cell lines in the repository are characterized and indexed as super donor cell lines via HLA mapping (e.g., HLA typing and/or matching). In addition, the compositions, systems, and methods disclose a fully indexed repository of iPSCs and/or iPS cell lines and/or cells derived therefrom that can be re-quantified and re-queried infinitely.

Because the HSC cells and/or cell lines, and/or blood progenitor cells and/or cell lines are characterized and indexed by HLA type, an HSC cell and/or cell line, and/or blood progenitor cell and/or line can be identified as suitable for a given patient with a compatible HLA type, with low, reduced, or zero chance of HSC transplant rejection. In certain embodiments, the bank of HSCs and/or blood progenitors is comprehensive in that it contains a variety of stem cells and/or stem cell lines (e.g., iPSCs and/or iPSC lines and/or cells derived therefrom), indexed by HLA types covering a significant proportion (e.g., at least 85%, at least 90%, or at least 95%) of a given population. In certain embodiments, the HSC lines and/or blood progenitors in the bank (and/or the iPS cell lines and/or embryoid bodies from which the HSCs and/or blood progenitors are derived), are characterized and indexed as super donor cell lines (e.g., via HLA mapping). Thus, it is possible to obviate the need for bone marrow registries, since suitable cells for transplantation may be quickly identified from the bank of iPSCs and/or iPSC lines and/or cells derived therefrom (e.g., HSCs and/or blood progenitors) and made available to patients over a wide swath of a given population upon demand, without the difficult, time consuming process of identifying a matching blood marrow donor. Identification of a suitable cell line may include matching the patient's ABO blood type and/or RHD blood group to that of the HSC, blood progenitor cell, embryoid body, and/or iPSC line, in addition to HLA type.

The bank may provide access to reserves of immortalized iPSCs from which HSCs and/or blood progenitors can be derived—HSCs and/or blood progenitors may be prepared in advance for commonly-used/matched HLA types (e.g., HLA superdonors matching higher percentages of the population) so that cells are available immediately upon need. HSCs may also be produced for a particular patient upon identification of a matching iPSC line from the HLA-indexed bank of stem cells and/or cell lines. Furthermore, in certain embodiments, reserves of embryoid bodies, corresponding to characterized and indexed iPSC lines, are stored in the HLA-indexed bank. In certain embodiments, HLA superdonor lines are physically represented in the bank by embryoid bodies (characterized and indexed as HLA superdonor lines). These embryoid bodies may be used to make HSCs and/or blood progenitors.

In one aspect, the invention is directed to a method of querying and retrieving data entries of a HLA-indexed database that match a set of queried human leukocyte antigen (HLA) loci for identification, production, and/or retrieval of hematopoietic stem cells (HSCs) suitable for treatment of a subject, said method comprising the steps of: storing, by a processor of a computing device (e.g., a server), the database comprising a data entry corresponding to each of a plurality of characterized induced pluripotent stem cell (iPSC) lines (e.g., or corresponding embryoid bodies) (e.g., wherein iPS cells for each of the characterized iPSC lines can be used to form embryoid bodies for each iPSC line and/or hematopoietic stem cells (HSCs) and/or blood progenitors for each iPSC line, e.g., wherein iPS cells and/or embryoid bodies and/or HSCs and/or blood progenitors derived from/corresponding to each of the characterized iPSC lines are stored in a physical repository), the data entry for each of the plurality of characterized iPSC lines (e.g., or corresponding embryoid bodies) comprising a set of characterized HLA loci corresponding to the iPSC line [e.g., each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci]; receiving, by the processor, a query from a user, the query comprising the set of queried HLA loci for the subject; and retrieving, by the processor, one or more data entries of the database, each representative of an iPSC line (e.g., and/or an embryoid body and/or an HSC line and/or a blood progenitor line derived from an iPSC line (e.g., cells in the physical repository)) matching (e.g., exactly matching, partially matching, identified as compatible with (e.g., compatible HLA types), etc.) the set of queried HLA loci (e.g., determining the corresponding bar code or other identifier for the cells corresponding each of the retrieved matching data entries, thereby allowing retrieval of desired cells from the repository and/or retrieval of identifying information corresponding to a desired cell line matching the queried HLA loci).

In certain embodiments, each of the one or more retrieved data entries of the database is representative of an iPSC cell line in a repository and/or a cell line in the repository corresponding to (e.g., derived from) an iPSC cell line (e.g., a member selected from the group consisting of an embryoid body, an HSC, a mesenchymal stem cell (MSC), a retinal pigment epithelium (RPE), a blood progenitor, a chondrocyte, a neuron, and a cardiomyocyte). In certain embodiments, each of the iPSC lines corresponding to the one or more data entries of the database is stored in a physical repository.

In certain embodiments, the set of characterized HLA loci corresponding to each of the plurality of characterized iPSC lines comprises each of a set of at least 3 given loci, wherein the at least 3 given loci are HLA-A, HLA-B, and HLA-DRB. In certain embodiments, the set of characterized HLA loci corresponding to each of the plurality of characterized iPSC lines comprises at least 9 given loci, wherein the at least 9 given loci are HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci corresponding to each of the plurality of characterized iPSC lines comprises at least 3 given loci (e.g., at least 3, at least 4, at least 5, at least 6, at least 7, at least 8, or at least 9) selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the method comprises retrieving one or more cells (e.g., iPSCs and/or embryoid bodies and/or blood progenitor cells and/or HSCs) from the physical repository corresponding to the one or more retrieved data entries.

In certain embodiments, the data entry corresponding to each of a plurality of characterized induced pluripotent stem cell (iPSC) lines further comprises ABO blood type and the query further comprises ABO blood type (in addition to the HLA loci), and wherein the retrieving step comprises retrieving, by the processor, one or more data entries, each representative of the iPSC line matching the set of queried HLA loci and the queried ABO blood type.

In certain embodiments, the data entry corresponding to each of a plurality of characterized induced pluripotent stem cell (iPSC) lines further comprises RHD blood group and the query further comprises RHD blood group, and wherein the retrieving step comprises retrieving, by the processor, one or more data entries, each representative of the iPSC line matching the queried RHD blood group and the set of queried HLA loci (e.g., and the queried ABO blood type).

In certain embodiments, the set of queried HLA loci correspond to the subject in need of an HLA match [e.g., the HLA match corresponding to one or more samples represented in the database (e.g., each sample comprising cells, e.g., IPS cells and/or embryoid bodies and/or blood progenitors and/or HSCs and/or iPSC-derived cells) exactly matching, partially matching, identified as compatible with (e.g., compatible HLA type), etc., the HLA loci of the subject]. In certain embodiments, the HLA match is each of the iPSC lines corresponding to each of the one or more retrieved data entries of the database that exactly match or partially match the set of queried HLA loci of the subject.

In certain embodiments, the set of queried HLA loci, and/or ABO blood type, and/or RHD blood group is determined by processing and analyzing a biological sample from a subject in need of an HLA match and/or an ABO match and/or an RHD match.

In certain embodiments, the method further comprises retrieving characterized cells from the physical repository, wherein the characterized cells correspond to the one or more retrieved data entries matching the set of queried HLA loci.

In certain embodiments, the method further comprises producing blood progenitors and/or HSCs from an iPSC line corresponding to the one or more retrieved data entries matching the set of queried HLA loci.

In certain embodiments, the method further comprises administering blood progenitors and/or HSCs to the subject, wherein said blood progenitors and/or HSCs are derived from/produced from an iPSC line (e.g., and/or an embryoid body corresponding to/produced from the iPSC line) corresponding to the one or more retrieved data entries matching the set of queried HLA loci.

In certain embodiments, the method comprises administering the blood progenitors and/or the HSCs to the subject for treatment of a known disease or condition in the subject, wherein the known disease or condition is a member selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

In certain embodiments, the database comprises a data entry corresponding to each of a plurality iPSC super donor cell lines [e.g., wherein the embryoid body cell line or super donor cell line can be used for treatment of multiple subjects without (or with lower risk of) rejection, e.g., wherein the HLA loci in the database for each cell line is a super donor type (e.g., the HLA type for each cell line does not trigger strong immune rejection reactions in multiple subjects, and/or the HLA type for each cell line comprises homozygous HLA gene combinations], wherein the data entry for each super donor cell line comprises a set of characterized HLA loci corresponding to the super donor cell line [e.g., identification (e.g., by processing and analyzing (e.g. by serology, by PCR) samples from an individual (e.g., blood samples)) of each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci].

In certain embodiments, each of the plurality of the iPSC super donor cell lines are used for treatment of the subject with lower risk of immune rejection by the subject. In certain embodiments, the method comprises determining the set of characterized HLA loci corresponding to each of the plurality of the super donor cell lines by processing and analyzing one or more biological samples collected from each of one or more super donor individuals. In certain embodiments, the step of determining the set of characterized HLA loci corresponding to each of the plurality of the super donor cell lines comprises identifying a set of at least 3 HLA loci, wherein the at least 3 HLA loci are HLA-A, HLA-B, and HLA-DRB. In certain embodiments, the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci corresponding to each of the plurality of the super donor cell lines comprises at least 3 (e.g., at least 4, at least 5, at least 6, at least 7, at least 8, or at least 9) HLA loci selected from the group consisting of HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci corresponding to each of the plurality of the super donor cell lines comprises at least 3 homozygous HLA loci selected from the group consisting of HLA-A, HLA-B, and DRB. In certain embodiments, the homozygous set of characterized HLA loci belong to a set of most-common HLA loci for a given population that matches a majority of the given population (e.g. the U.S. population). In certain embodiments, the homozygous set of characterized HLA loci comprise homozygous HLA loci in at least 3 (e.g., at least 4, or at least 5, or at least 6, or at least 7, or at least 8, or at least 9) major sites, wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the plurality of iPSC super donor cell lines (e.g., or embryoid body cell lines) matches at least 70%, (e.g., at least 75%, at least 80%, at least 85%, at least 90%, or at least 95%) of the population from which the subject originates (e.g., the U.S. population).

In another aspect, the invention is directed to a system for querying and retrieving data entries of a database matching queried human leukocyte antigen (HLA) loci for identification and/or production and/or retrieval of blood progenitors or hematopoietic stem cells (HSCs) suitable for treatment of a subject, the system comprising: a repository of characterized embryoid bodies and/or characterized induced pluripotent stem cells (iPSCs) and/or characterized blood progenitor cells and/or characterized HSCs (e.g., said HSCs derived from iPSCs and/or embryoid bodies); a processor; and a non-transitory computer readable medium having instructions stored thereon, wherein the instructions, when executed by the processor, cause the processor to perform the steps of any one of the methods described herein.

In another aspect, the invention is directed to a system of querying and retrieving data entries of an HLA-indexed database that match a set of queried human leukocyte antigen (HLA) loci for identification, production, and/or retrieval of hematopoietic stem cells (HSCs) suitable for treatment of a subject, the system comprising: a physical repository comprising a plurality of cells corresponding to characterized induced pluripotent stem cell (iPSC) lines [e.g., wherein the cells are iPSC cells and/or cells corresponding to (e.g., derived from) iPSC cells, e.g., any one or more members selected from the group consisting of embryoid bodies, HSCs, mesenchymal stem cells (MSCs), retinal pigment epithelium (RPEs), blood progenitors, chondrocytes, neurons, and cardiomyocytes]; a processor; a non-transitory computer readable medium having instructions stored thereon, wherein the instructions, when executed by the processor, cause the processor to: store the database, said database comprising a data entry corresponding to each of the plurality of characterized iPSC lines in the physical repository, the data entry for each of the plurality of characterized iPSC lines comprising a set of characterized HLA loci corresponding to the iPSC line; receive a query from a user, the query comprising the set of queried HLA loci for the subject; and retrieve, by the processor, one or more data entries of the database, each representative of an iPSC line matching the set of queried HLA loci (e.g., an iPSC line stored in the physical repository).

In certain embodiments, each of the one or more retrieved data entries of the database is representative of an iPSC cell line in the physical repository and/or a cell line in the physical repository corresponding to an iPSC cell line. In certain embodiments, the set of characterized HLA loci corresponding to each of the plurality of characterized iPSC lines comprises each of a set of at least 3 given loci, wherein the at least 3 given loci are HLA-A, HLA-B, and HLA-DRB. In certain embodiments, the set of characterized HLA loci corresponding to each of the plurality of characterized iPSC lines comprises at least 9 given loci, wherein the at least 9 given loci are HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci corresponding to each of the plurality of characterized iPSC lines comprises at least 3 (e.g., at least 3, at least 4, at least 5, at least 6, at least 7, at least 8, or at least 9 members are selected from the at least 9 given loci) given loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, each of the one or more retrieved data entries of the database exactly match or partially match the set of queried HLA loci for the subject. In certain embodiments, the data entry corresponding to each of the plurality of characterized induced pluripotent stem cell (iPSC) lines further comprises ABO blood type and the query further comprises ABO blood type, and wherein the processor retrieves one or more data entries, each representative of the iPSC line matching the set of queried HLA loci and the queried ABO blood type. In certain embodiments, the data entry corresponding to each of the plurality of characterized induced pluripotent stem cell (iPSC) lines further comprises RHD blood group and the query further comprises RHD blood group, and wherein the processor retrieves one or more data entries, each representative of the iPSC line matching the queried RHD blood group and the set of queried HLA loci.

In certain embodiments, the set of queried HLA loci correspond to the subject in need of an HLA match. In certain embodiments, the HLA match is each of the iPSC lines corresponding to each of the one or more retrieved data entries of the database that exactly match or partially match the set of queried HLA loci of the subject.

In certain embodiments, blood progenitors and/or HSCs are administered to the subject treatment of a known disease or condition in the subject, wherein the known disease or condition is a member selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency. In certain embodiments, wherein the physical repository comprises one or more liquid nitrogen storage tanks (e.g., and/or another freezer system).

In certain embodiments, the database comprises a data entry corresponding to each of a plurality of iPSC super donor cell lines, wherein the data entry for each super donor cell line comprises a set of characterized HLA loci corresponding to the super donor cell line. In certain embodiments, each of the plurality of the iPSC super donor cell lines can be used for treatment of the subject with lower risk of immune rejection by the subject.

In certain embodiments, the set of characterized HLA loci corresponding to each of the plurality of the super donor cell lines comprises a set of at least 3 HLA loci, wherein the at least 3 HLA loci are HLA-A, HLA-B, and HLA-DRB. In certain embodiments, the set of characterized HLA loci corresponding to each of the plurality of the super donor cell lines comprises a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the set of characterized HLA loci corresponding to each of the plurality of the super donor cell lines comprises at least 3 homozygous HLA loci selected from the group consisting of HLA-A, HLA-B, and DRB. In certain embodiments, the homozygous set of characterized HLA loci belong to a set of most-common HLA loci for a given population that matches a majority of the given population. In certain embodiments, the homozygous set of characterized HLA loci comprise homozygous HLA loci in at least 3 major sites (e.g., or at least 4, or at least 5, or at least 6, or at least 7, or at least 8, or at least 9 major sites), wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1. In certain embodiments, the plurality of iPSC super donor cell lines match at least 70%, (e.g., at least 75%, at least 80%, at least 85%, at least 90%, or at least 95%) of the population from which the subject originates.

In certain embodiments, the plurality of cells corresponding to characterized iPSC lines in the physical repository are capable of being cultured, expanded, stored, partially and/or fully differentiated, and transferred to a subject (e.g., a subject with matching HLA loci, wherein the transferred cells do not induce a significant immune rejection, wherein the transfer requires lower doses of immunosuppressant drugs to be administered to the subject, and wherein the transferred cells do not cause significant infiltration by cells of the immune system in the subject).

In certain embodiments, the physical repository is a biorepository for collecting, processing, storing, and/or distributing biospecimens, wherein each of the biospecimens is a member selected from the group consisting of iPSCs, iPSC-derived cells, and tissue created from iPSCs.

In certain embodiments, the physical repository is in communication with one or more processors programmed for identifying, locating, and/or inventorying the biospecimens in the physical repository. In certain embodiments, the physical repository is outfitted with hardware and/or robotics for automated sample handling.

In certain embodiments, the system comprises characterized super donor cells.

In certain embodiments, the system is used for one or more clinical procedures. In certain embodiments, each of the one or more clinical procedures is a member selected from the group consisting of gene therapy, cell transplant, and tissue transplant.

In certain embodiments, the system is used for one or more pre-clinical studies. In certain embodiments, each of the one or more pre-clinical studies is a member selected from the group consisting of in vitro screens, in vivo screens, efficacy testing of medications, toxicity testing of medications, and testing for use in personalized medicine.

In another aspect, the invention is directed to a method of treating a subject, the method comprising: administering blood progenitors and/or HSCs to the subject, said blood progenitors and/or HSCs derived from/produced from an iPSC line (and/or an embryoid body corresponding to/produced from the iPSC line) corresponding to the one or more retrieved data entries matching the set of queried HLA loci, wherein iPS cells from the iPSC line (and/or the embryoid body corresponding to/produced from the iPSC line) are stored in and retrieved from the physical repository of any one of the system and methods described herein.

In another aspect, the invention is directed to a repository of characterized cells and/or cell lines (e.g., undifferentiated cells (e.g., induced pluripotent stem cells (iPSCs)), differentiated cells (e.g., hair cells, fibroblasts, blood cells) that are capable of being cultured (e.g., in vitro, in vivo), expanded (e.g., in vitro, in vivo), stored (e.g., frozen), partially un/differentiated (e.g., into progenitor cells), differentiated (e.g., into tissue-specific cells (e.g., cardiomyocytes, hepatocytes) into blood cells, neurons), and transferred to a recipient (e.g., a human subject in need of such differentiated cells), wherein the transferred cells do not induce significant immune rejection (e.g., the transferred cells are not destroyed in the recipient (e.g. by the recipient's immune system), transferring requires lower doses of immunosuppressant drugs, the transferred cells do not cause significant infiltration by cells of the immune system (e.g., T cells, eosinophils, plasma cells, neutrophils) in the recipient (e.g., wherein the repository is a biorepository for collecting, processing, storing, and/or distributing biospecimens, e.g., biological samples, iPSCs, and/or cells or tissue created from iPSCs, e.g., wherein the repository is in electrical communication with one or more processors programmed for identifying, locating, and/or inventorying biospecimens in the repository, e.g., wherein the repository is outfitted with hardware, robotics, etc., for automated sample handling).

In certain embodiments, the cells and/or cell lines are characterized by human leukocyte antigen (HLA) mapping of at least 3, at least 4, at least 5, at least 6, at least 7, at least 8, or at least 9 HLA loci, wherein the HLA loci are members selected from the group consisting of HLA-A, HLA-B, HLA-C, HLA-DRB1, HLA-DRB3, HLA-DRB4, HLA-DRB5, HLA-DQB1, HLA-DPB1. In certain embodiments, the cells and/or cell lines are characterized by HLA allele locus (e.g., A, B, C, DR, DQ, and/or DP) and individual specificity (e.g., A1, B27, DR8, etc.).

In certain embodiments, the characterized cells and/or cell lines are characterized by ABO blood type. In certain embodiments, the characterized cells and/or cell lines are characterized by RHD group. In certain embodiments, the repository comprises the characterized cells and/or cell lines that are homozygous in at least 3 HLA loci (e.g., HLA-A, HLA-B, and HLA-DRB (e.g., HLA-DRB1)).

In certain embodiments, the repository comprises the characterized cells and/or cell lines that are homozygous in at least 3, at least 4, at least 5, at least 6, at least 7, at least 8, or at least 9 HLA loci, wherein the HLA loci are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the repository comprises characterized super donor cells and/or cell lines (e.g., the super donor cell lines can be used for treatment of multiple subjects without rejection, the HLA loci for each cell line is a super donor type (e.g., the HLA type for each cell line does not trigger strong immune rejection reactions in multiple subjects, the HLA type for each cell line comprises homozygous HLA gene combinations).

In certain embodiments, the homozygous HLA gene combinations comprise homozygous HLA-A, HLA-B, and DRB-1 combinations. In certain embodiments, the homozygous HLA-A, HLA-B, and DRB-1 combinations comprise one of the top ranking homozygous HLA-A, HLA-B, and DRB-1 combinations for a given population. In certain embodiments, the homozygous HLA gene combinations comprise homozygous HLA loci in at least 3, at least 4, at least 5, at least 6, at least 7, at least 8, or at least 9 major sites, wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the repository comprises the characterized cells and/or cell lines that are derived from one or more biological samples of one or more individual donors. In certain embodiments, each of the characterized cells and/or cell lines in the repository have corresponding data comprising a set of characterized HLA loci of the cells and/or cell line, said corresponding data being stored in a searchable database for retrieval of one or more matching (e.g., exactly matching, partially matching, identifying as compatible with (e.g., compatible HLA types), and the like with a queried HLA loci for the subject)) physical cell lines upon query.

Elements of embodiments involving one aspect of the invention (e.g., methods) can be applied in embodiments involving other aspects of the invention (e.g., systems), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

“Sample” or “Biological Sample”: The term “sample” or “biological sample”, as used herein, refers to a biological sample obtained or derived from a source of interest, as described herein. In certain embodiments, a source of interest comprises an organism, such as a microbe, a plant, an animal or a human. In certain embodiments, a biological sample is or comprises biological tissue or fluid. In certain embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In certain embodiments, a biological sample is or comprises cells obtained from an individual. In certain embodiments, obtained cells are or include cells from an individual from whom the sample is obtained. In certain embodiments, a sample is a “primary sample” obtained directly from a source of interest by any appropriate means. For example, in certain embodiments, a primary biological sample is obtained by methods selected from the group consisting of a swab, biopsy (e.g., fine needle aspiration or tissue biopsy), surgery, collection of body fluid (e.g., blood, lymph, feces etc.), etc. In certain embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc.

“Genotyping data”: As used herein, the term “genotyping data” refers to data obtained from measurements of a genotype. In certain embodiments, genotyping data describes an individual's phenotype. Genotyping data may be measurements of particular genes (e.g., portions of an individual's genetic sequence, e.g., DNA sequence), SNPs, or variants of SNPs. In certain embodiments, genotyping data is obtained from a multi-gene panel. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

“Partially un/differentiated”: As used herein, the term “partially un/differentiated” describes a biological cell that, like a state of stem cell, has a tendency to differentiate into a specific type of cell, but is already more specific than a stem cell and is pushed to differentiate into its “target” cell. For example, a difference between stem cells and progenitor cells is that stem cells can replicate indefinitely, whereas progenitor cells can divide only a limited number of times. An example of a partially un/differentiated cell is a progenitor cell.

“Subject” or “Individual”: As used herein, the term “subject” or “individual” refers to a human or other animal, or plant. In certain embodiments, subjects are humans and mammals (e.g., mice, rats, pigs, cats, dogs, horses, and primates). In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals are, for example, rodents (e.g., mice, rats, hamsters), rabbits, primates, or swine such as inbred pigs and the like.

“Bank”: As used herein, the term “bank” refers to a system, apparatus, or location where genetic material and/or biological sample is stored. Genetic material may be derived (e.g., extracted) from a biological sample provided by an individual to the organization that owns and/or operates the bank. In certain embodiments, biological samples are stored in a bank separate from a bank that stores genetic material extracted therefrom.

“Reserve”: As used herein, the term “reserve” refers to an amount of biological material (e.g., cells and/or cell lines) stored in a bank.

“Variant”: As used herein, the term “variant” refers to a specific variation of a specific SNP occurring in the genome of an organism. In certain embodiments, a variant is a specific combination of a first allele of a first copy of an individual's genetic material (e.g., corresponding to an individual's paternal DNA) and a second allele of a second copy of an individual's genetic material (e.g., corresponding to an individual's maternal DNA), as occurs in diploid organisms (e.g., humans).

“Cells” or “Cell lines”: As used herein, the term “cells” or “cells lines” refers to cells derived from human and/or non-human samples. In certain embodiments, cells can include in vitro cultured cells like iPSC-derived cells. In certain embodiments, cells can include cell lines. For example, cells can include iPSCs, and/or hematopoietic stem cells (HSCs), and/or blood progenitor cells, and/or mesenchymal stem cells (MSCs), and/or Retinal Pigment Epithelium (RPEs), and/or chondrocytes, and/or embryoid bodies, and/or any other iPSC-derived cells, and/or iPSC lines, and/or HSC lines, and/or blood progenitor cell lines, and/or MSCs lines, and/or RPE lines, and/or chondrocyte lines, and/or embryoid bodies of an iPSC line, and/or any other iPSC-derived cell lines. The cells and/or cell lines may or may not be immortalized.

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting.

### DESCRIPTION OF THE DRAWINGS

The Drawings, which are comprised of at least the following Figures, is for illustration purposes only, not for limitation.

FIG. 1 shows an illustrative network environment 100 for use in the methods and systems described herein.

FIG. 2 shows an example of a computing device 200 and a mobile computing device 250 that can be used in the methods and systems described in this disclosure.

FIG. 3 is a block diagram of a method of querying and retrieving data entries of a HLA-indexed database that match queried human leukocyte antigen (HLA) loci for treatment of a subject, according to an illustrative embodiment of the invention.

FIG. 4 is a block diagram of a method of treating a subject, according to an illustrative embodiment of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Described herein is a managed, HLA-indexed repository of hematopoietic stem cell (HSC) lines and/or blood progenitor cell lines derived from induced pluripotent stem cells (iPSCs) (or embryoid bodies formed from iPSCs), wherein each of the HSC lines, blood progenitor cell lines, MSC lines, embryoid bodies, and/or iPSC lines has corresponding data comprising a set of characterized HLA loci, said corresponding data being stored in a searchable database for retrieval of one or more matching physical cell lines upon query. The repository comprises a bank of iPSCs, embryoid bodies, HSCs, and/or blood progenitor cells for each of a set of indexed HLA types, for identification and provision of allogeneic cell lines suitable for transplantation to reestablish hematopoietic function in patients with damaged, diseased, or otherwise abnormal bone marrow and/or immune system.

### Human Leukocyte Antigen (HLA)

The characterized iPS cells and/or cell lines stored in the repository are indexed using the Human Leukocyte Antigen (HLA). In certain embodiments, the iPS cells and/or cell lines and/or cells derived therefrom are characterized and indexed as super donor cell lines via HLA mapping (e.g., HLA typing and/or matching). In certain embodiments, multiple HLA loci may be characterized and indexed for each of the various iPS cells and/or cell lines and/or cells derived therefrom.

The HLAs in humans are major histocompatibility complex (MHC) proteins that function to regulate the immune system. HLA genes are highly polymorphic and may be broadly divided into Class I and Class II. For example, Class I in humans may be found on all nucleated cells and platelets. On the other hand, HLA Class II (constitutive expression), for example, may be restricted to specialized cells of the immune system (e.g., macrophages, B cells, etc.).

HLA Class I, for example, may include HLA-A, B, and C genes. In certain embodiments, HLA Class I may be co-dominantly expressed on the cell surface and may present peptides derived from internal cellular proteins to the T cell receptor of CD8 T cells. For example, these proteins may be involved in the immune response against intracellular parasites, viruses, and cancer.

In certain embodiments, HLA Class I may have a heterodimeric protein structure, with a polymorphic alpha chain and a common beta-2 microglobulin. In certain embodiments, the alpha chain may be composed of 3 extracellular domains: α1, α 2, and α 3.

HLA Class II, for example, may include DR, DQ, and DP genes. In certain embodiments, HLA Class II may be co-dominantly expressed. In certain embodiments, HLA Class II may have a heterodimeric protein structure, with a polymorphic beta chain and a much less polymorphic alpha chain. In certain embodiments, both chains may be composed of two (2) extracellular domains (α1, α2, and β1, β2). For example, the α1 and β1 domains may create a peptide binding groove which presents processed peptides, from extracellular protein, to CD4+ T cells. In certain embodiments, HLA Class II may be involved in the immune response against extracellular infectious agents and non-self HLA molecules.

In certain embodiments, each HLA allele may be identified by letters indicating “locus” (e.g., A, B, C, DR, DQ, and DP) and individual specificity may be defined by a number following the locus (e.g., A1, B27, DR8, etc.). Specificities can be defined using antisera (antibodies). In certain embodiments, HLA specificities may also be determined using genetic analysis by identifying the presence/absence of the gene encoding the HLA protein. For example, Class II molecular specificities may be identified at the level of the gene encoding a particular chain (α or β).

### HLA Typing

The stem cells and/or stem cell lines (e.g., iPSCs, and/or cells derived therefrom) indexed and stored in the physical repository may be characterized and indexed using various characteristics of the samples (e.g., cells). In certain embodiments, the stems cells and/or cell lines and/or cells derived therefrom may be characterized and indexed using HLA type. In certain embodiments, the stems cells and/or cell lines and/or cells derived therefrom may be characterized and indexed using ABO blood group. In certain embodiments, the stems cells and/or cell lines and/or cells derived therefrom may be characterized and indexed using RHD blood type. For example, the stems cells and/or cell lines and/or cells derived therefrom may be characterized and indexed in the physical repository using HLA type, and/or ABO blood group, and/or RHD type.

HLA typing or HLA matching is used to determine the HLA type of an individual. The HLA type of an individual comprises a pair of co-expressed haplotypes, each corresponding to a set of HLA genes (e.g., an HLA-A, an HLA-B, and an HLA-DR gene). In certain embodiments, genetic recombination and environmental factors result in linkage disequilibrium with respect to inheritance of HLA gene combinations. For example, certain combinations of HLA alleles (e.g., combinations of HLA-A, -B, and -DR genes) are favored, whereas other combinations do not exist.

HLA typing may be performed at a protein level but may also be performed at the DNA level, for example by amplifying the DNA via polymerase chain reaction (PCR), or other DNA identification and amplification technologies. For example, HLA typing may be performed using sequence specific oligonucleotides (SSO). In certain embodiments, SSO-based HLA typing may use generic primers to amplify large amounts of HLA alleles, for example, HLA-A, via PCR or other DNA amplification technologies. The dsDNA is separated into single strands and allowed to interact with the single strand specific oligonucleotide probes. In certain embodiments, such probes may be bound to a solid matrix. For example, the pattern of the bound probes may be used to determine the HLA type of the specimen. In certain embodiments, HLA typing may be performed using sequence specific primers (SSP). For example, in SSP-based HLA typing amplifies DNA that matches the primers. Antibodies may also been used for HLA typing, but may have the disadvantage of cross-reacting with multiple HLA epitopes (e.g. HLA-A2, A9 and A28).

### Applications of HLA Typing

The HLA type of a sample (e.g., cells, organs, and/or tissue) may be used in determining compatibility between organ donors and recipients. Samples which match the HLA type of a recipient (e.g., patient) are more likely to not illicit an immune response (e.g., rejection) after the sample is transplanted to the recipient. In certain embodiments, matching is performed on the basis of 3 or more loci on the HLA gene to prevent a strong immune response in the recipient post transplantation. In certain embodiments, at least 3 HLA loci are required to match between the donor and the recipient to prevent a strong immune response in the recipient post transplantation. In certain embodiments, at least 3, or at least 4, or at least 5, at least 6, or at least 7, or at least 8, or at least 9 major sites (e.g., loci) are required to match between the donor and the recipient to prevent a strong immune response in the recipient post transplantation.

Many registry donors have been tested by serological (e.g., HLA mapping using antigens) methods, though often without documentation regarding which antigens were tested. While the majority of hematopoietic progenitor cell transplant candidates have been tested by molecular (DNA-based) methodologies, the nomenclature of antigens (serology) and alleles (DNA) is in some cases not concordant. Thus, the characterized and indexed (e.g., HLA indexed (e.g., using standard nomenclature)) iPS cells and/or cell lines and/or cells derived therefrom, described herein, may be used to efficiently and accurately searched using the corresponding database to quickly find matching HLA samples for implantation. For example, the HLA indexed and matched iPS cells and/or cell lines and/or cells derived therefrom may be used in treatment of various diseases. In certain embodiments, these cells and/or cell lines may be used in the treatment cancer (e.g., leukemia, lymphoma, bone cancer, and the like). In certain embodiments, these cells and/or cell lines may be used in Hematopoietic stem cell transplantation.

The HLA-indexed repository may also be used for various purposes. For example, other clinical applications of HLA typing may include disease risk assessment, pharmacogenomics, immunotherapy, infectious disease vaccines, and tumor vaccines. In certain embodiments, the cells and/or cell lines stored and indexed in the repository may be used in cosmetic surgery, for example cartilage grafts. Long-term transplant and graft survival is correlated to the degree of HLA antigen mismatch for both solid organ and bone marrow transplant.

HLA matched cells and/or cell lines may also be used in the treatment of various diseases. Certain diseases may have a strong association with certain specific HLA types. For example, HLA associations with diseases include ankylosing spondylitis and acute anterior uveitis (HLA-B27); birdshot retinopathy (HLA-A29); Behçet's Disease (HLA-B51); psoriasis (HLA-Cw6); celiac disease (HLA-DQ2,8); narcolepsy (HLA-DR15, DQ6); diabetes (HLA-DR3,4-DQ2,8); and rheumatoid arthritis (HLA-DR4). In certain embodiments, the data entries in the HLA database corresponding to specific samples (e.g., cells and/or cell lines in the physical repository) may incorporate information regarding their specific HLA types to recognize their strong associations with certain diseases.

HLA type may also be associated with allergy or hypersensitivity to a medication. For example, severe allergic or hypersensitivity reaction to drugs in Stevens-Johnson Syndrome (SJS) and toxic epidermal necrolysis (TEN) may be associated with HLA type. The physical repository of cells and/or cells lines and corresponding database may be used to identify allergies and sensitivities in the patients (e.g., sometimes unknown to the patient). In certain embodiments, HLA typing allows risk stratification of the patients. In certain embodiments, drugs that are associated with hypersensitivity reactions (e.g., antiepileptic agents, allopurinol, nevirapine, anti-inflammatories in oxicam family, and sulfonamides) may be studied using the cells and/or cell lines and/or cells derived therefrom stored in the repository. Further, these studies can be performed in vitro and/or ex vivo prior to implantation.

HLA typing may be used for vaccine development. The HLA-indexed cells and/or cell lines and/or cells derived therefrom described herein may be used to develop such vaccines. In certain embodiments, vaccines producing cellular immunity require peptide HLA binding. For example, vaccine trials use peptides binding to common HLA alleles. After proof-of-principal, trials may include peptides binding to other HLA alleles. In certain embodiments, cells with the common HLA allele, and cells with other HLA alleles may be selected from the back of stem cells and/or cell lines stored in the repository.

HLA typing can also be informative for compatibility of individuals. For example, studies have found that husbands and wives have fewer HLA matches than expected. The HLA genes (HLA-A, HLA-B, and HLA-DRB1) regulate the immune system, and thus determine the microbes that the immune system attacks. As a non-limiting example, the HLA genes therefore regulate a subject's smell by governing the non-human microbes associated with that subject and therefore can affect the attraction between subjects based on smell, among other things. Given the association between HLA type and long-term compatibility, it may be possible to predict the likelihood of companionship between two individuals. In some embodiments, the present disclosure teaches a method of querying and retrieving data entries of a database matching queried HLA loci for compatibility or companionship for a given subject with other individuals.

HLA-Indexed Induced Pluripotent Stem Cell (iPSC) Bank

The bank of IPS cells and/or cell lines and/or cells derived therefrom (e.g., HSCs and/or blood progenitors) is a comprehensive repository of in that it contains a variety of HLA types covering a significant proportion (e.g., at least 85%, at least 90%, or at least 95%) of a given population, indexed by HLA type and/or ABO group and/or RHD type. In certain embodiments, the HSC lines and/or blood progenitors in the bank (and/or the iPS cell lines and/or embryoid bodies from which the HSCs and/or blood progenitors are derived), may be characterized as super donor cell lines (e.g., via HLA mapping). Thus, it is possible to obviate the need for bone marrow registries and/or other donor registries, since suitable cells for transplantation may be quickly identified and made available to patients over a wide swath of a given population upon demand, without the difficult, time consuming process of identifying a matching blood marrow donor. Identification of a suitable cell line may include matching the patient's ABO blood type and/or RHD blood group to that of the HSC, blood progenitor cell, embryoid body, and/or iPSC line, in addition to HLA type.

The bank may provide access to reserves of immortalized iPSCs from which HSCs and/or blood progenitors can be derived—HSCs and/or blood progenitors may be prepared in advance for commonly-used/matched HLA types (e.g., HLA superdonors matching higher percentages of the population) so that cells are available immediately upon need. HSCs may also be produced for a particular patient upon identification of a matching iPSC line. Furthermore, in certain embodiments, reserves of embryoid bodies, corresponding to characterized iPSC lines, are stored in the bank. In certain embodiments, HLA superdonor lines are physically represented in the bank by embryoid bodies (characterized as HLA superdonor lines). These embryoid bodies may be used to make HSCs and/or blood progenitors.

Induced human pluripotent stem cells (iPSCs) can be generated from biological samples, such as blood samples. Depending on the conditions, in vitro iPSCs can retain their pluripotency or they can be directed to differentiate into a wide range of specialized cell types and tissues. Such cell types and tissues can be used for applications including replacement of diseased or damaged tissues in patients with conditions such as trauma, diabetes, degenerative neurological disorders, cardiovascular disease, and metabolic deficiencies.

As discussed in Taylor et al., Cell Stem Cell 11, Aug. 3, 2012, pp. 147-152, incorporated herein by reference, HLA-mismatched iPSCs can cause immunological rejection and therefore limit therapeutic potential. iPSCs derived directly from patients (autologous iPSCs) can result in matched HLA type and reduce risk of transplant rejection. However, generation of autologous iPSCs for individual patients is costly and time-consuming. Alternatively, allogeneic iPSC cell lines with HLA types that do not trigger strong reactions can be prepared and used for large groups of individuals.

The term “super donor” is a term used to describe HLA types that do not trigger strong rejection reactions. Such allogeneic (derived from donors other than the patient) iPSC lines can be made in advance and can be ready for use when needed. Fewer allogeneic lines are needed to serve a population. iPSCs can be obtained from healthy volunteer donors of blood group O that are selected to maximize the opportunity for HLA matching. Clinical grade iPSC lines can be expanded and differentiated for use in a large number of subjects. Nakajima et al., Stem Cells 25, 2007, pp. 983-985, which is hereby incorporated by reference herein, discusses HLA matching estimations in a hypothetical bank of human embryonic stem cell lines in the Japanese population, and calculated that a large proportion of patients were able to find at least one HLA matched donor at three loci of HLA-A, HLA-B, and HLA-DR for transplantation therapy.

As iPSC-derived cell lines like HSC lines and/or blood progenitor cell lines are characterized by HLA type, an HSC line and/or blood progenitor cell line can be identified as suitable for a given patient with a compatible HLA type, with low, reduced, or zero chance of HSC transplant rejection. The characterized iPSCs and/or embryoid bodies comprising embryonic stem cells (e.g., undifferentiated pluripotent cells) can be differentiated into hematopoietic cells such as HSCs, hematopoietic progenitor cells, and mature hematopoietic cells in the presence of appropriate culture media. In certain embodiments, the characterized cell types contained in the physical bank include any one or more of the following: iPSCs, embryoid bodies, HSCs, blood progenitor cells, and/or mature hematopoietic cells.

Matching HLA type may involve, for example, querying and retrieving data entries of a database matching queried HLA loci. In certain embodiments, this comprises receiving, by a processor of a computing device (e.g., a server), a data entry for an individual for which a matching HSC line is desired, the data entry comprising a set of characterized HLA loci corresponding to the individual [e.g., identification (e.g., by processing and analyzing (e.g. by serology, by PCR) samples from the individual (e.g., blood samples)) of each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci]; and retrieving, by the processor, one or more data entries of a database representative of cells (e.g., iPS cells in the physical repository and/or HSCs from a cell line derived from iPSCs) matching (e.g., exactly matching, partially matching, identified as compatible with (e.g., compatible HLA types), etc.) the queried HLA loci (e.g., determining the corresponding bar code or other identifier for the iPSCs and/or HSCs corresponding to the data entry, thereby allowing retrieval of desired hematopoietic stem cells from the repository and/or retrieval of identifying information corresponding to a desired HSC and/or iPSC cell line matching the queried HLA loci. HSCs may be produced from immortalized iPSC lines at will and made available for ready access when needed—no additional harvesting of samples are required to produce additional HSCs.

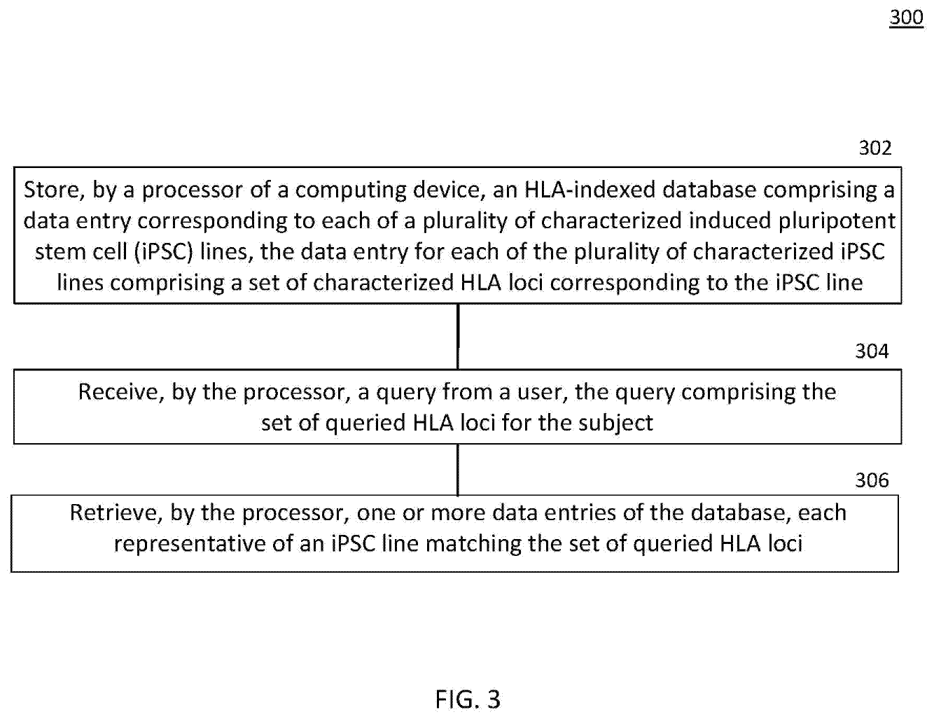

FIG. 3. is a block diagram of a method 300 of querying and retrieving data entries of a HLA-indexed database that match a set of queried human leukocyte antigen (HLA) loci for treatment of a subject. In step 302, the processor of a computing device stores the database comprising a data entry corresponding to each of the plurality of characterized stem cells (e.g., iPSCs, embryoid bodies, HSCs, MSCs, RPEs, and/or blood progenitor cells) and/or cell lines (e.g., iPSC lines, HSC lines, MSC lines, RPE lines, and/or blood progenitor cell lines) or corresponding embryoid bodies and/or cells derived therefrom. In certain embodiments, the characterized stem cells may include iPSCs, HSCs, RPEs, blood progenitor cells, and MSCs. In certain embodiments, the characterized stem cell lines may include iPSC lines, HSC lines, RPE lines, blood progenitor lines, and MSC lines. In certain embodiments, the cells derived therefrom may include iPSC-derived cardiomyocytes, iPSC-derived neurons, iPSC-derived chondrocytes, and the like. The data entry for each iPSC, and/or iPSC line, and/or corresponding embryoid bodies, and/or cells derived therefrom (e.g., HSCs, MSCs, RPEs, blood progenitor cells, cardiomyocytes, neurons, chondrocytes, and the like) comprises a set of characterized HLA loci corresponding to the iPSC line the cells are derived from. In step 304, the processor of the computing device receives, from a user a query, the query comprising a set of queried HLA loci for the subject. In certain embodiments, the set of queried loci comprises at least 3 HLA loci. In certain embodiments, the set of queried loci comprises at least 3, at least 4, at least 5, at least 6, at least 7, at least 8, or at least 9 HLA loci. The processor of the computing device then retrieves (306) one or more data entries of the database, each representative of an iPSC line and/or an embryoid body and/or an HSC line and/or a blood progenitor line and/or an MSC line and/or an RPE line derived from an iPSC line matching the set of queried HLA loci.

The repository/bank of cells may comprise a storage system comprising an insulated container equipped with environmental control system (for control of temperature, humidity, pressure, and the like) suitable to store cells (e.g., iPSCs, embryoid bodies, HSCs, blood progenitor cells, MSCs, RPEs, chondrocytes, cardiomyocytes and/or mature hematopoietic cells) for a period of time. The repository/bank may also include one or more processors (e.g., of a server) and/or related software to manage inventory, as well as a sample location system and/or retrieval system for identification/retrieval of cells from a matched cell line. iPSCs may be produced from blood samples (or other biological substance sample, e.g., saliva, serum, tissue, cheek cells, cells collected via a buccal swab, urine, and/or hair), then labeled (physically and/or digitally), logged in an inventory database, and stored in the repository for ongoing and/or future use. HSCs may be produced from iPSCs via known methods, and the HSCs may also be labeled (physically and/or digitally), logged in the inventory database, and stored in the repository for ongoing and/or future use.

The repository/bank of cells may be used in systems and methods for treatment of subjects in need of bone marrow transplants. For example, the repository/bank of cells comprise iPSCs and/or embryoid bodies corresponding to/produced from iPSC lines, wherein HSCs and/or blood progenitors are derived from/produced from the iPSCs and/or embryoid bodies, and the HSCs and/or blood progenitors are administered to subjects at risk of or having a disease and/or condition, such as any of the following: acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

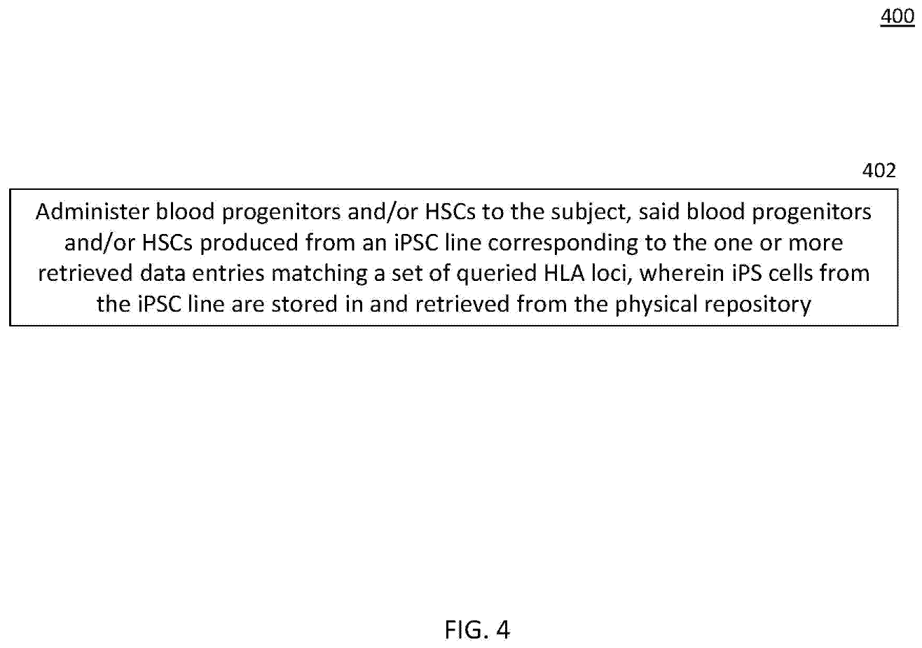

FIG. 4 is a block diagram showing a method 400 of treating a subject. In step 402, blood progenitors and/or HSCs are administered to the subject, said blood progenitors and/or HSCs produced from an iPSC line corresponding to the one or more retrieved data entries matching the set of queried HLA loci, wherein iPS cells from the iPSC line are stored in and retrieved from the physical repository described herein. In certain embodiments, the blood progenitors and/or HSCs administered to the subject are produced from embryoid bodies corresponding to the iPSC line that corresponds to the one or more retrieved data entries matching the set of queried HLA loci.

Generation and Differentiation Protocols for Immortalized iPSCs

Induced pluripotent stem cell (iPSC) generation protocols are described, for example, at https://www.thermofisher.com/us/en/home/references/protocols/cell-culture/stem-cell-protocols/ipsc-protocols.html, the contents of which is hereby incorporated by reference in its entirety. Induced pluripotent stem cell (iPSC) generation and differentiation protocols are described, for example, at http://www.sigmaaldrich.com/life-science/stem-cell-biology/ipsc/ipsc-protocols.html, the contents of which is hereby incorporated by reference in its entirety. Differentiation of iPSCs can be found, for example, in “Induction of Pluripotent Stem Cells from Adult Human Fibroblasts by Defined Factors”; Takahashi K., Tanabe K., Ohnuki M., Narita M., Ichisaka T., Tomoda K., Yamanaka S.; Cell Vol. 131, 861-872, November 2007″, the contents of which is hereby incorporated by reference in its entirety.

Recently, HSCs have been successfully produced from iPSCs. See, for example, “Generation of engraftable hematopoietic stem cells from induced pluripotent stem cells by way of teratoma formation,” Mol Ther. 2013 July; 21(7); 1424-31; Epub May 14, 2013; “Hematopoietic stem cells meet induced pluripotent stem cells technology,” Haematologica, 2016 September; 101(9): 999-1001; and “In vivo generation of transplantable human hematopoietic cells from induced pluripotent stem cells,” Blood, 2013 Feb. 21; 121(8); 1255-64; Epub Dec. 4, 2012; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; the contents of each of which are incorporate herein by reference.

Storage of Immortalized iPSCs

Repositories (290) (e.g., cell repositories, e.g., nucleic acid repositories) for storing biological sample material (e.g., cells, e.g., nucleic acids) can include liquid nitrogen storage tanks and/or other freezer systems. Liquid nitrogen tanks provide temperature (e.g., about −195° C.) and/or humidity control, and can be used to store, for example, immortalized cell lines (e.g., immortalized iPSCs) over a long period of time. Alternatively, biological material (e.g., nucleic acids) can be stored in freezer systems at higher temperatures (e.g., from about −80° C. to about −20° C.). Additional equipment, backup systems, software/inventory control systems, sample location systems, automated sample retrieval, etc. can be used for storage and/or maintenance of the biological sample material stored in the repositories. The described setup allows for backup systems (e.g., additional repositories) to be used if a given tank and/or freezer temperature control system and/or humidity control system malfunctions.

Moreover, the provided systems and methods can record and track, via a graphical user interface, biological samples (and biological material extracted therefrom) used to generate genotyping data, for example, as described in U.S. Application No. 62/485,778, entitled “Chain Of Custody For Biological Samples And Biological Material Used In Genotyping Tests” and filed on Apr. 14, 2017, U.S. application Ser. No. 15/846,659 entitled “Chain Of Custody For Biological Samples And Biological Material Used In Genotyping Tests” filed on Dec. 19, 2017, and International Application No. PCT/US17/67272 entitled “Chain of Custody for Biological Samples and Biological Material Used in Genotyping Tests” filed on Dec. 19, 2017, the contents of which are hereby incorporated by reference in their entirety.

For example, as biological samples are processed in several stages to extract biological material and perform genotyping tests, IDs are assigned to biological sample material for individuals as well as well plates used during processing of the biological sample material in order to organize the samples and the tests. Biological sample materials are assigned to well plates for use in extracting biological material. Biological sample material is assigned to genotyping plates for use in performing genotyping tests. By associating IDs corresponding to biological sample material with IDs for well plates or genotyping plates, respectively, a user can track which extractions and/or tests need to be performed as well as record which biological samples have been received or genotyping plates analyzed via a graphical user interface.

### Illustrative Computer Network Environment

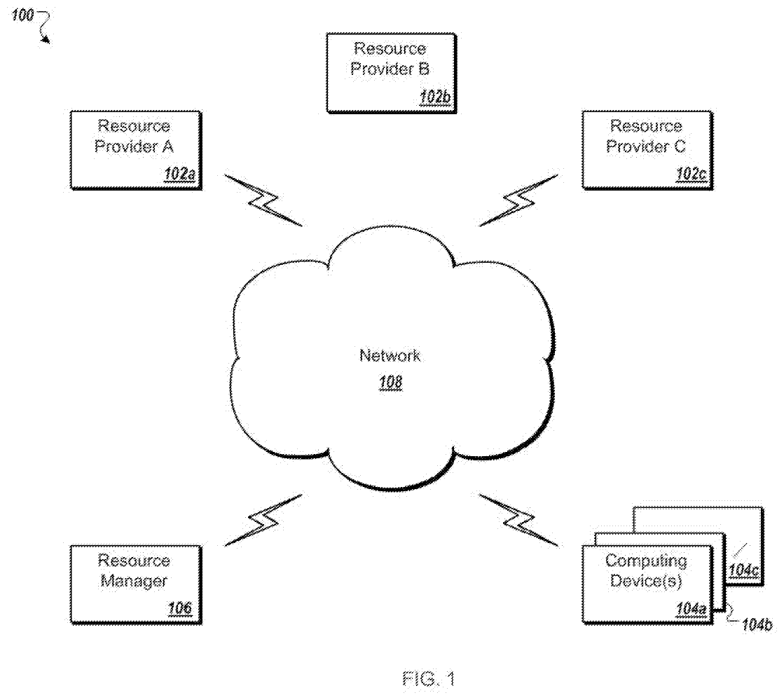

FIG. 1 shows an illustrative network environment 100 for use in the methods and systems described herein. In brief overview, referring now to FIG. 1, a block diagram of an exemplary cloud computing environment 100 is shown and described. The cloud computing environment 100 may include one or more resource providers 102a, 102b, 102c (collectively, 102). Each resource provider 102 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 102 may be connected to any other resource provider 102 in the cloud computing environment 100. In some implementations, the resource providers 102 may be connected over a computer network 108. Each resource provider 102 may be connected to one or more computing device 104a, 104b, 104c (collectively, 104), over the computer network 108.

The cloud computing environment 100 may include a resource manager 106. The resource manager 106 may be connected to the resource providers 102 and the computing devices 104 over the computer network 108. In some implementations, the resource manager 106 may facilitate the provision of computing resources by one or more resource providers 102 to one or more computing devices 104. The resource manager 106 may receive a request for a computing resource from a particular computing device 104. The resource manager 106 may identify one or more resource providers 102 capable of providing the computing resource requested by the computing device 104. The resource manager 106 may select a resource provider 102 to provide the computing resource. The resource manager 106 may facilitate a connection between the resource provider 102 and a particular computing device 104. In some implementations, the resource manager 106 may establish a connection between a particular resource provider 102 and a particular computing device 104. In some implementations, the resource manager 106 may redirect a particular computing device 104 to a particular resource provider 102 with the requested computing resource.

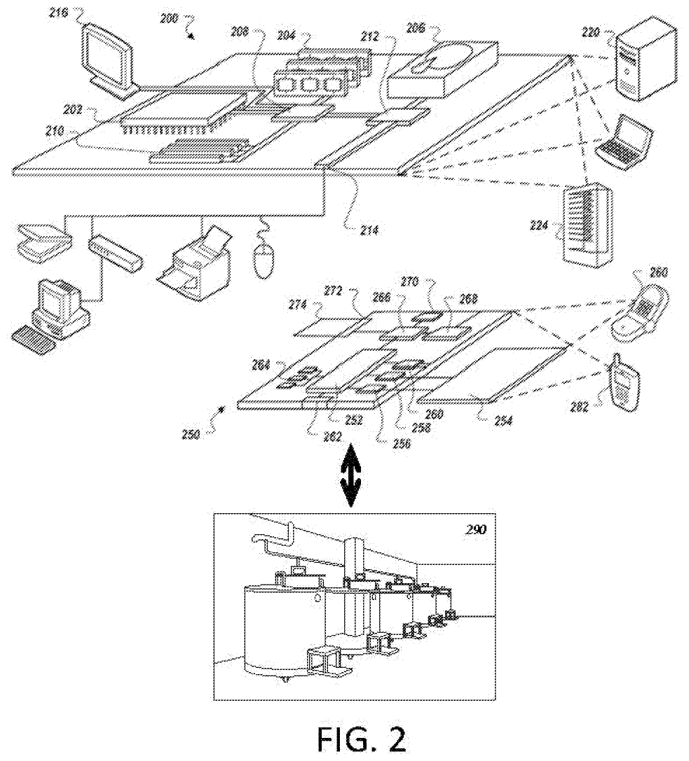

FIG. 2 shows an example of a computing device 200 and a mobile computing device 250 that can be used in the methods and systems described in this disclosure. The computing device 200 is intended to represent various forms of digital computers, such as laptops, desktops, workstations, personal digital assistants, servers, blade servers, mainframes, and other appropriate computers. The mobile computing device 250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 200 includes a processor 202, a memory 204, a storage device 206, a high-speed interface 208 connecting to the memory 204 and multiple high-speed expansion ports 210, and a low-speed interface 212 connecting to a low-speed expansion port 214 and the storage device 206. Each of the processor 202, the memory 204, the storage device 206, the high-speed interface 208, the high-speed expansion ports 210, and the low-speed interface 212, are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as a display 216 coupled to the high-speed interface 208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 204 stores information within the computing device 200. In some implementations, the memory 204 is a volatile memory unit or units. In some implementations, the memory 204 is a non-volatile memory unit or units. The memory 204 may also be another form of computer-readable medium, such as a magnetic or optical disk.

The storage device 206 is capable of providing mass storage for the computing device 200. In some implementations, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 202), perform one or more methods, such as those described above. The instructions can also be stored by one or more storage devices such as computer- or machine-readable mediums (for example, the memory 204, the storage device 206, or memory on the processor 202).

The high-speed interface 208 manages bandwidth-intensive operations for the computing device 200, while the low-speed interface 212 manages lower bandwidth-intensive operations. Such allocation of functions is an example only. In some implementations, the high-speed interface 208 is coupled to the memory 204, the display 216 (e.g., through a graphics processor or accelerator), and to the high-speed expansion ports 210, which may accept various expansion cards (not shown). In the implementation, the low-speed interface 212 is coupled to the storage device 206 and the low-speed expansion port 214. The low-speed expansion port 214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 220, or multiple times in a group of such servers. In addition, it may be implemented in a personal computer such as a laptop computer 222. It may also be implemented as part of a rack server system 224. Alternatively, components from the computing device 200 may be combined with other components in a mobile device (not shown), such as a mobile computing device 250. Each of such devices may contain one or more of the computing device 200 and the mobile computing device 250, and an entire system may be made up of multiple computing devices communicating with each other.

The mobile computing device 250 includes a processor 252, a memory 264, an input/output device such as a display 254, a communication interface 266, and a transceiver 268, among other components. The mobile computing device 250 may also be provided with a storage device, such as a micro-drive or other device, to provide additional storage. Each of the processor 252, the memory 264, the display 254, the communication interface 266, and the transceiver 268, are interconnected using various buses, and several of the components may be mounted on a common motherboard or in other manners as appropriate.

The processor 252 can execute instructions within the mobile computing device 250, including instructions stored in the memory 264. The processor 252 may be implemented as a chipset of chips that include separate and multiple analog and digital processors. The processor 252 may provide, for example, for coordination of the other components of the mobile computing device 250, such as control of user interfaces, applications run by the mobile computing device 250, and wireless communication by the mobile computing device 250.

The processor 252 may communicate with a user through a control interface 258 and a display interface 256 coupled to the display 254. The display 254 may be, for example, a TFT (Thin-Film-Transistor Liquid Crystal Display) display or an OLED (Organic Light Emitting Diode) display, or other appropriate display technology. The display interface 256 may comprise appropriate circuitry for driving the display 254 to present graphical and other information to a user. The control interface 258 may receive commands from a user and convert them for submission to the processor 252. In addition, an external interface 262 may provide communication with the processor 252, so as to enable near area communication of the mobile computing device 250 with other devices. The external interface 262 may provide, for example, for wired communication in some implementations, or for wireless communication in other implementations, and multiple interfaces may also be used.

The memory 264 stores information within the mobile computing device 250. The memory 264 can be implemented as one or more of a computer-readable medium or media, a volatile memory unit or units, or a non-volatile memory unit or units. An expansion memory 274 may also be provided and connected to the mobile computing device 250 through an expansion interface 272, which may include, for example, a SIMM (Single In Line Memory Module) card interface. The expansion memory 274 may provide extra storage space for the mobile computing device 250, or may also store applications or other information for the mobile computing device 250. Specifically, the expansion memory 274 may include instructions to carry out or supplement the processes described above, and may include secure information also. Thus, for example, the expansion memory 274 may be provided as a security module for the mobile computing device 250, and may be programmed with instructions that permit secure use of the mobile computing device 250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 252), perform one or more methods, such as those described above. The instructions can also be stored by one or more storage devices, such as one or more computer- or machine-readable mediums (for example, the memory 264, the expansion memory 274, or memory on the processor 252). In some implementations, the instructions can be received in a propagated signal, for example, over the transceiver 268 or the external interface 262.

The mobile computing device 250 may communicate wirelessly through the communication interface 266, which may include digital signal processing circuitry where necessary. The communication interface 266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 268 using a radio-frequency. In addition, short-range communication may occur, such as using a Bluetooth®, Wi-Fi™, or other such transceiver (not shown). In addition, a GPS (Global Positioning System) receiver module 270 may provide additional navigation- and location-related wireless data to the mobile computing device 250, which may be used as appropriate by applications running on the mobile computing device 250.

The mobile computing device 250 may also communicate audibly using an audio codec 260, which may receive spoken information from a user and convert it to usable digital information. The audio codec 260 may likewise generate audible sound for a user, such as through a speaker, e.g., in a handset of the mobile computing device 250. Such sound may include sound from voice telephone calls, may include recorded sound (e.g., voice messages, music files, etc.) and may also include sound generated by applications operating on the mobile computing device 250.

The mobile computing device 250 may be implemented in a number of different forms, as shown in the figure. For example, it may be implemented as a cellular telephone 280. It may also be implemented as part of a smart-phone 282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In certain embodiments, the system comprises a physical biorepository 290 (comprising one or more cell storage containers) in communication with any of the computer system arrangements of FIG. 1 or 2.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Documents are incorporated herein by reference as noted. Where there is any discrepancy in the meaning of a particular term, the meaning provided in the Definition section above is controlling.

Certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description. Therefore, the disclosure should not be limited to certain implementations, but rather should be limited only by the spirit and scope of the claims.

### Exemplification

### Example 1: HLA-Indexed Cell Repository (LifeCapsule)

The present example describes an HLA-indexed cell repository, as well as methods for generating an HLA-indexed cell repository of this type. Cells in this repository can be transformed into iPSCs that can be cultured, expanded, differentiated, stored, and transferred into one or more subjects for whom the iPSCs have been determined to be compatible.

Blood samples were collected from volunteer donors. These blood samples were collected, for example, via crowdsourcing and mail-in blood samples. Collected blood samples were processed and analyzed (e.g., using PCR, e.g., using whole genome sequencing). Further, data associated with each sample including HLA genotype at different loci, e.g., nine (9) different loci, was determined. Loci that were validated include: HLA-A, HLA-B, HLA-C, HLA-DRB1, HLA-DRB3, HLA-DRB4, HLA-DRB5, HLA-DQB1, HLA-DPB1. Other information determined and validated for each sample included ABO blood type and RHD.

The data determined for each sample was then stored in a living database. A living database is a database that is updated with new information as and when determined. The data stored for each sample includes, for example, but is not limited to, date of collection, attributed diseases, medical identifier number, name, contact information, date of birth, date of sample collection, geographical location, family groups, current medication, HLA information, ABO information, and RHD information. Each of these is stored as a field in the database, and may be arranged in a tree structure. For example, the medical identifier number is a unique name tag that identifies the sample in the database and may be associated with further information of the sample donor including but not limited to the donor's name, contact information, and date of birth. The living database is continually updated with sample data collected from additional volunteer donors.

For example, a subset of the entries of a living database is presented in Table 1 below. The living database entries in Table 1 include the following data for each sample: Batch identification number, Client identification number, HLA haplotype information, Blood type (ABO) information, and RhD (RHD) information, and sample type. The HLA haplotype information in this database includes haplotype information for nine (9) different loci. The loci that were haplotyped and validated include: HLA-A, HLA-B, HLA-C, HLA-DRB1, HLA-DRB3, HLA-DRB4, HLA-DRB5, HLA-DQB1, HLA-DPB1. Moreover, genotyping information of both alleles are determined and stored for each sample in the database. For example, for the HLA-A loci, genotype information for each allele, labeled A1 and A2, are stored for each sample in the database. Samples that are homozygous at various HLA loci were also identified and stored. For example, a sample that is homozygous for a particular HLA locus (e.g., HLA-A) was stored in the living database with the first allele field of the data entry (e.g., A1) storing the genotype of the locus, and the second allele field of the data entry (e.g., A2) storing a (a hyphen). For example, the sample with Client ID # B528007 of Table 1 is homozygous in HLA-A. Furthermore, samples that did not contain certain HLA loci (e.g., the less common DPB1 locus) were identified. For these samples, the corresponding fields of the data entry (e.g., DPB11 and DPB12) in the living database stored an ‘x’. Samples represented in the living database can be stored in multiple forms in the cell repository.

Each entry in the living database (e.g., corresponding to each row of Table 1) is associated with or corresponds to each original blood sample collected from each donor and a reserve of one or more physical cells is stored in a bank. Each blood sample collected from each donor is characterized (e.g., by HLA typing, ABO typing, RHD typing), frozen and stored. A portion of each blood sample is also transformed into an iPSC. The iPSCs are cultured in vitro, expanded, and frozen. These iPSCs can be differentiated into other cell or tissue types (e.g., HSCs, MSCs, cardiomyocytes, nerve cells, skin cells, etc.). These HLA-indexed iPSCs and/or the differentiated cells and/or tissues may be transferred (e.g., stem cell transplantation) into subjects (e.g., humans (e.g., who are a match (e.g., HLA match, ABO match, and/or RHD match))) in need of stem cell treatment and/or transplantation without the need for extensive wait times.

The complete living database contains data entries for cell lines from at least 69 super donors found to be homozygous for at least 3 significant loci (A, B, DRB (e.g., any one of the DRB genes)). Entries of the living database corresponding to these 69 super donor cell lines are presented in Table 2. Of the 69 super donor cell lines in the living database, at least 12 are homozygous in at least 6 loci. Furthermore, of the 69 super donor cell lines in the living database, at least 31 have 0 blood type which is the universal donor blood type. Furthermore, the living database contains HLA compatible matches for at least 90% of the US population (see constructive Example 2 below). Additionally, the living database contains HLA compatible matches for other populations, including Japan and the U.K, although the percentage of compatible matches were smaller for these populations as HLA types and their occurrence in a population can vary by ethnicity.

TABLE 1
An example of a living Database

| Batch Id | Client Id | A1 | A2 | B1 | B2 | C1 | C2 | DRB11 | DRB12 | DRB31 | DRB32 |
|---|---|---|---|---|---|---|---|---|---|---|---|
| PT0000003 | AB548067 | 30:01:01G | 68:02:01G | 07:02:01G | 42:01:01 | 07:01:01G | 17:01:01G | 3:02:01 | 11:01:02 | 01:01:02G | 02:02:01G |
| PT0000005 | B528004 | 01:01:01G | 03:01:01G | 41:01:01 | 57:01:01G | 06:02:01G | 17:01:01G | 03:01:01G | 4:04:01 | 01:01:02G | — |
| PT0000005 | B528005 | 01:01:01G | 02:01:01G | 15:01:01G | 58:01:01G | 03:03:01G | 07:01:01G | 4:01:01 | 07:01:01G | x | x |
| PT0000005 | B528006 | 01:01:01G | 02:01:01G | 15:01:01G | 58:01:01G | 03:03:01G | 07:01:01G | 4:01:01 | 07:01:01G | x | x |
| PT0000005 | B528007 | 02:07:01G | — | 15:01:01G | 46:01:01G | 01:02:01G | 04:01:01G | 8:03:02 | 09:01:02G | x | x |
| PT0000005 | B528009 | 01:01:01G | 24:02:01G | 08:01:01G | 37:01:01G | 06:02:01G | 07:01:01G | 03:01:01G | 14:01:01G | 01:01:02G | 02:02:01G |
| PT0000005 | B528010 | 02:01:01G | 26:01:01G | 13:02:01G | 15:01:01G | 06:02:01G | 07:04:01G | 07:01:01G | 11:04:01 | 02:02:01G | — |
| PT0000005 | B528013 | 01:01:01G | 03:01:01G | 07:02:01G | 52:01:01G | 07:02:01G | 12:02:01G | 15:01:01G | 15:02:01 | x | x |
| PT0000005 | B528014 | 02:01:01G | 11:01:01G | 35:01:01G | 44:02:01G | 04:01:01G | 05:01:01G | 1:03 | 11:01:01G | 02:02:01G | — |
| PT0000005 | B528018 | 01:01:01G | 03:01:01G | 08:01:01G | 27:05:02G | 01:02:01G | 07:01:01G | 01:01:01G | 03:01:01G | 01.01:02G | — |
| PT0000005 | B528022 | 33:03:01G | — | 58:01:01G | — | 03:02:01G | — | 4:04:01 | 13:02:01 | 03:01:01G | — |
| PT0000005 | B528024 | 02:01:01G | — | 14:02.01 | 18:01:01G | 07:01:01G | 08:02:01G | 08:01:01G | 13:02:01 | 03:01:01G | — |
| PT0000005 | B528031 | 02:01:01G | 33:01:01G | 15:01:01G | 44:02:01G | 03:03:01G | 05:01:01G | 01:01:01G | 07:01:01G | x | x |
| PT0000005 | B528032 | 01:01:01G | 33:01:01G | 07:02:01G | 14:02:01 | 07:02:01G | 08:02:01G | 03:01:01G | 15:01:01G | 02:02:01G | — |
| PT0000005 | B528034 | 01:01:01G | 34:02:01 | 08:01:01G | 35:01:01G | 07:01:01G | 16:01:01G | 03:01:01G | 07:01:01G | 01:01:02G | — |
| PT0000005 | B528035 | 01:01:01G | 68:01:02G | 08:01:01G | 15:01:01G | 03:03:01G | 07:01:01G | 03:01:01G | 4:04:01 | 01:01:02G | — |
| PT0000004 | B529001 | 02:01:01G | 03:01:01G | 14:02:01 | 44:02:01G | 05:01:01G | 08:02:01G | 4:01:01 | 13:02:01 | 03:01:01G | — |
| PT0000004 | B529002 | 02:01:01G | 32:01:01G | 42:01:01 | 73:01:00 | 15:05:01G | 17:01:01G | 8:04:01 | 13:02:01 | 03:01:01G | — |
| PT0000004 | B529004 | 02:07:01G | 11:01:01G | 40:01:01G | 46:01:01G | 01:02:01G | 03:04:01G | 09:01:02G | 12:02:01 | 03:01:01G | — |
| PT0000004 | B529005 | 31:01:02G | 66.01.01G | 38:01:01 | 41:02:01 | 12.03.01G | 17:01:01G | 4:02:01 | 11:01:01G | 02.02.01G | — |
| PT0000004 | B529007 | 02:07:01G | 11:01:01G | 46:01:01G | 55:12:00 | 01:02:01G | — | 12:02:01 | — | 03:01:01G | — |
| PT0000004 | B529008 | 01:01:01G | 31:01:02G | 27:05:02G | 37:01:01G | 05:01:01G | 06:02:01G | 01:01:01G | 15:01:01G | x | x |
| PT0000004 | B529009 | 24:02:01G | 31:01:02G | 07:02:01G | 51:01:01G | 07:02:01G | 15:02:01G | 4:08:01 | 13:01:01G | 01:01:02G | — |
| PT0000004 | B529010 | 01:01:01G | 03:01:01G | 07:02:01G | 08:01:01G | 07:01:01G | 07:02:01G | 03:01:01G | 11:01:01G | 01:01:02G | 02:02:01G |
| PT0000004 | B529011 | 01:01:01G | 02:01:01G | 35:01:01G | 44:02:01G | 04:01:01G | 05:01:01G | 03:01:01G | 4:01:01 | 01:01:02G | — |
| PT0000004 | B529012 | 23:01:01G | — | 08:01:01G | 44:03:01G | 04:01:01G | 07:01:01G | 07:01:01G | 15:01:01G | x | x |
| PT0000004 | B529013 | 02:01:01G | 68:01:01G | 08:01:01G | 55:01:01G | 01:02:01G | 07:01:01G | 03:01:01G | 14:07:01 | 01:01:02G | 02:02:01G |
| PT0000004 | B529014 | 24:02:01G | 29:02:01G | 44:03:01G | 55:01:01G | 07:02:01G | 16:01:01G | 03:01:01G | 04:07:01G | 02:02:01G | — |
| PT0000004 | B529019 | 02:01:01G | 32:01:01G | 14:01:01 | 57:01:01G | 06:02:01G | 08:02:01G | 4:04:01 | 07:01:01G | x | x |
| PT0000004 | B529020 | 30:01:01G | 68:02:01G | 07:02:01G | 42:01:01 | 07:02:01G | 17:01:01G | 03:01:01G | 11:02:01 | 02:02:01G | — |
| PT0000004 | B529023 | 02:01:01G | 32:01:01G | 18:01:01G | 51:01:01G | 01:02:01G | 07:01:01G | 11:04:01 | 16:01:01 | 02:02:01G | — |
| PT0000004 | B529039 | 02:01:01G | 24:02:01G | 35:01:01G | 44:02:01G | 04:01:01G | 05:01:01G | 1:03 | 4:01:01 | x | x |
| PT0000004 | B529041 | 02:01:01G | — | 39:01:01G | 44:03:01G | 07:02:01G | 16:01:01G | 07:01:01G | — | x | x |
| PT0000004 | B529043 | 24:02:01G | 32:01:01G | 13:02:01G | 47:01:01G | 06:02:01G | 07:01:01G | 01:01:01G | 07:01:01G | x | x |
| PT0000005 | B529048 | 02:01:01G | 32:01:01G | 07:02:01G | 44:03:01G | 04:01:01G | 07:02:01G | 4:02:01 | 07:01:01G | x | x |
| PT0000005 | B529049 | 11:01:01G | 24:02:01G | 18:01:01G | 40:01:01G | 03:04:01G | 07:01:01G | 4:03:01 | 15:01:01G | x | x |
| PT0000005 | B529053 | 02:01:01G | 02:05:01G | 13:02:01G | 57:01:01G | 06:02:01G | — | 07:01:01G | — | x | x |
| PT0000005 | B529055 | 01:01:01G | 02:05:01G | 14:02:01 | 50:01:01G | 06:02:01G | 08:02:01G | 1:02:01 | 07:01:01G | x | x |
| PT0000005 | B529059 | 01:01:01G | 02:05:01G | 13:02:01G | 57:01:01G | 06:02:01G | — | 11:01:01G | 16:01:01 | 02:02:01G | — |
| PT0000005 | B529060 | 01:01:01G | 24:02:01G | 15:17:01G | 37:01:01G | 06:02:01G | 07:01:01G | 09:01:02G | 13:02:01 | 03:01:01G | — |
| PT0000005 | B529061 | 11:01:01G | 32:01:01G | 07:02:01G | 50:01:01G | 06:02:01G | 07:02:01G | 07:01:01G | 15:01:01G | x | x |
| PT0000005 | B529062 | 02:01:01G | 11:01:01G | 08:01:01G | 35:01:01G | 04:01:01G | 07:01:01G | 01:01:01G | 15:01:01G | x | x |
| PT0000005 | B529065 | 01:01:01G | 02:01:01G | 08:01:01G | 15:01:01G | 03:03:01G | 07:01:01G | 03:01:01G | 13:01:01G | 01:01:02G | 02:02:01G |
| PT0000005 | B529068 | 01:01:01G | 03:01:01G | 14:01:01 | 35:01:01G | 07:02:01G | 08:02:01G | 07:01:01G | 14:01:01G | 02:02:01G | — |
| PT0000005 | B529069 | 33:01:01G | 33:03:01G | 14:02:01 | 58:01:01G | 03:02:01G | 08:02:01G | 1:02:01 | 13:02:01 | 03:01:01G | — |
| PT0000005 | B529074 | 02:01:01G | 03:01:01G | 07:02:01G | 44:02:01G | 05:01:01G | 07:02:01G | 07:01:01G | 08:01:01G | x | x |
| PT0000005 | B529079 | 23:01:01G | 33:11:01G | 08:01:01G | 14:02:01 | 07:01:01G | 08:02:01G | 03:01:01G | 14:01:01G | 01:01:02G | 02:02:01G |
| PT0000005 | B529081 | 01:01:01G | 02:01:01G | 07:02:01G | 08:01:01G | 07:01:01G | 07:02:01G | 03:01:01G | 15:01:01G | 01:01:02G | — |
| PT0000005 | B529089 | 11:01:01G | 29:02:01G | 35:01:01G | 49:01:01G | 04:01:01G | 07:01:01G | 07:01:01G | 13:02:01 | 03:01:01G | — |
| PT0000005 | B529093 | 24:02:01G | 26:01:01G | 14:02:01 | 35:02:01G | 04:01:01G | 08:02:01G | 1:02:01 | 11:04:01 | 02:02:01G | — |
| PT0000004 | B530001 | 02:01:01G | 29:02:01G | 35:01:01G | 44:03:01G | 04:01:01G | 16:01:01G | 07:01:01G | 14:01:01G | 02:01:01G | — |
| PT0000004 | B530002 | 24:02:01G | 26:01:01G | 44:03:02G | 51:01:01G | 07:01:01G | 16:02:01G | 4:01:01 | 11:04:01 | 02:02:01G | — |
| PT0000004 | B530003 | 02:01:01G | — | 44:02:01G | 51:01:01G | 05:01:01G | 07:01:01G | 4:01:01 | 13:01:01G | 02:02:01G | — |
| PT0000004 | B530004 | 11:01:01G | 24:02:01G | 35:01:01G | 38:02:01G | 03:03:01G | 03:04:01G | 15:01:01G | 15:02:01 | x | x |
| PT0000004 | B530005 | 01:01:01G | 33:03:01G | 57:01:01G | 58:01:01G | 03:02:01G | 06:02:01G | 07:01.01G | 13:02:01 | 03:01:01G | — |
| PT0000004 | B530006 | 01:01:01G | 31:01:02G | 08:01:01G | 27:05:02G | 01:02:01G | 07:01:01G | 4:04:01 | 13:01:01G | 01:01:02G | — |
| PT0000004 | B530007 | 01:01:01G | 25:01:01G | 08:01:01G | 18:01:01G | 07:01:01G | 12:03:01G | 03:01:01G | 15:01:01G | 01:01:02G | — |
| PT0000004 | B530009 | 11:01:01G | 29:02:01G | 40:01:01G | 44:03:01G | 03:04:01G | 16:01:01G | 01:01:01G | 4:03:01 | x | x |
| PT0000004 | B530010 | 33:03:01G | — | 50:01:01G | 58:01:01G | 03:02:01G | 06:02:01G | 03:01:01G | 4:03:01 | 02:02:01G | — |
| PT0000004 | B530011 | 01:01:01G | — | 08:01:01G | — | 07:01:01G | — | 03:01:01G | — | 01:01:02G | — |
| PT0000004 | B530012 | 02:01:01G | 32:01:01G | 35:01:01G | 44:02:01G | 04:01:01G | 05:01:01G | 11:01:01G | — | 02:02:01G | — |
| PT0000004 | B530013 | 02:01:01G | 29:02:01G | 44:03:01G | 51:01:01G | 01:02:01G | 16:01:01G | 07:01:01G | 13:01:01G | 01:01:02G | — |

| Batch Id | Client Id | DRB41 | DRB42 | DRB51 | DRB52 | DQB11 | DQB12 | DPB11 | DPB12 | ABO | RHD | Sample Type |
|---|---|---|---|---|---|---|---|---|---|---|---|---|
| PT0000003 | AB548067 | x | x | x | x | 02:01:01G | 04:02:01G | 01:01:01G | — | O | + | Misc |
| PT0000005 | B528004 | 01:01:01G | — | x | x | 02:01:01G | 04:02:01G | 04:01:01G | — | A | + | Ext DNA |
| PT0000005 | B528005 | 01:01:01G | — | x | x | 02:01:01G | 03:02:01G | 02:01:02G | 04:02:01G | O | + | Ext DNA |
| PT0000005 | B528006 | 01:01:01G | — | x | x | 02:01:01G | 03:02:01G | 02:01:02G | 04:02:01G | O | + | Ext DNA |
| PT0000005 | B528007 | 01:01:01G | — | x | x | 03:03:02G | 06:01:01G | 02:01:02G | 05:01:01G | B | + | Ext DNA |
| PT0000005 | B528009 | x | x | x | x | 02:01:01G | 05:03:01G | 01:01:01G | 03:01:01G | O | + | Ext DNA |
| PT0000005 | B528010 | 01:01:01G | — | x | x | 02:01:01G | 03:01:01G | 04:02:01G | 10:01 | AB | + | Ext DNA |
| PT0000005 | B528013 | x | x | 1:01:01 | 01:02:01G | 06:01:01G | 06:02:01G | 02:01:02G | — | O | + | Ext DNA |
| PT0000005 | B528014 | x | x | x | x | 03:01:01G | 05:01:01G | 02:01:02G | 04:01:01G | O | + | Ext DNA |
| PT0000005 | B528018 | x | x | x | x | 02:01:01G | 05:01:01G | 01:01:01G | 04:02:01G | O | + | Ext DNA |
| PT0000005 | B528022 | 01:01:01G | — | x | x | 03:02:01G | 06:09.01G | 05:01:01G | — | O | + | Ext DNA |
| PT0000005 | B528024 | x | x | x | x | 04:02:01G | 06:09:01G | 04:01:01G | 05:01.01G | A | + | Ext DNA |
| PT0000005 | B528031 | 01:01:01G | — | x | x | 02:01:01G | 05:01:01G | 04:01:01G | 11:01:01 | A | + | Ext DNA |
| PT0000005 | B528032 | x | x | 1:01:01 | — | 02:01:01G | 06:02:01G | 02:01:02G | 04:01:01G | A | + | Ext DNA |
| PT0000005 | B528034 | 01:01:01G | — | x | x | 02:01:01G | — | 01:01:01G | 17:01:01G | B | + | Ext DNA |
| PT0000005 | B528035 | 01.01:01G | — | x | x | 02:01:01G | 03:02:01G | 04:01:01G | — | O | + | Ext DNA |
| PT0000004 | B529001 | 01:01:01G | — | x | x | 03:01:01G | 06:09:01G | 05:01:01G | 14:01:01 | O | + | Ext DNA |
| PT0000004 | B529002 | x | x | x | x | 04:02:01G | 06:09:01G | 04:02:01G | 11:01:01 | O | + | Ext DNA |
| PT0000004 | B529004 | 01:01:01G | — | x | x | 03:01:01G | 03:03:02G | 02:01:02G | 14:01:01 | A | + | Ext DNA |
| PT0000004 | B529005 | 01:01:01G | — | x | x | 03.01:01G | 03:02:01G | 02:01:02G | 04:01:01G | O | + | Ext DNA |
| PT0000004 | B529007 | x | x | x | x | 03:01:01G | 05:02:01G | 02:01:02G | 05:01:01G | O | + | Ext DNA |
| PT0000004 | B529008 | x | x | 1:01:01 | — | 05:01:01G | 06:02:01G | 04:01:01G | — | AB | + | Ext DNA |
| PT0000004 | B529009 | 01:01:01G | — | x | x | 03:01:01G | 06:03:01G | 04:01:01G | 04:02:01G | AB | + | Ext DNA |
| PT0000004 | B529010 | x | x | x | x | 02:01:01G | 03:01:01G | 02:01:02G | 04:01:01G | AB | + | Ext DNA |
| PT0000004 | B529011 | 01:01:01G | — | x | x | 02:01:01G | 03:01:01G | 01:01:01G | 04:02:01G | A | + | Ext DNA |
| PT0000004 | B529012 | 01:01:01G | — | x | x | 02:01:01G | 06:02:01G | 04:02:01G | 17:01:01G | B | + | Ext DNA |
| PT0000004 | B529013 | x | x | x | x | 02:01:01G | 05:03:01G | 01:01:01G | 04:01:01G | A | + | Ext DNA |
| PT0000004 | B529014 | 01:01:01G | — | x | x | 02:01:01G | 03:01:01G | 03:01:01G | 04:01:01G | A | − | Ext DNA |
| PT0000004 | B529019 | 01:01:01G | — | x | x | 03:02:01G | 03:03:02G | 03:01:01G | 04:01:01G | O | + | Ext DNA |
| PT0000004 | B529020 | x | x | x | x | 02:01:01G | 03:01:01G | 04:02:01G | — | O | + | Ext DNA |
| PT0000004 | B529023 | x | x | 02:02:01G | — | 03:01:01G | 05:02:01G | 04:01:01G | — | O | + | Ext DNA |
| PT0000004 | B529039 | 01:01:01G | — | x | x | 03:01:01G | 05:01:01G | 02:01:02G | 04:01:01G | A | + | Ext DNA |
| PT0000004 | B529041 | 01:01:01G | — | x | x | 02:01:01G | 03:03:02G | 04:02:01G | 11:01:01 | B | + | Ext DNA |
| PT0000004 | B529043 | 01:01:01G | — | x | x | 02:01:01G | 05:01:01G | 14:01:01 | 17:01:01G | B | + | Ext DNA |
| PT0000005 | B529048 | 01:01:01G | — | x | x | 02:01:01G | 03:02:01G | 04:01:01G | 04:02:01G | A | + | Ext DNA |
| PT0000005 | B529049 | 01:01:01G | — | 1:01:01 | — | 03:02:01G | 06:02:01G | 04:01:01G | 05:01:01G | O | + | Ext DNA |
| PT0000005 | B529053 | 01:01:01G | — | x | x | 02:01:01G | 03:03:02G | 04:01:01G | — | A | + | Ext DNA |
| PT0000005 | B529055 | 01:01:01G | — | x | x | 02:01:01G | 05:01.01G | 02:01:02G | 03:01:01G | O | + | Ext DNA |
| PT0000005 | B529059 | x | x | 02:02:01G | — | 03:01:01G | 05:02:01G | 02:01:02G | 04:02:01G | O | + | Ext DNA |
| PT0000005 | B529060 | 01:01:01G | — | x | x | 03:03:02G | 06:04:01G | 02:01:02G | 14:01:01 | A | + | Ext DNA |
| PT0000005 | B529061 | 01:01:01G | — | 1:01:01 | — | 02:01:01G | 06:02:01G | 02:01:02G | 04:02:01G | A | + | Ext DNA |
| PT0000005 | B529062 | x | x | 1:01:01 | — | 05:01:01G | 06:02:01G | 04:01:01G | 10:01 | O | + | Ext DNA |
| PT0000005 | B529065 | x | x | x | x | 02:01:01G | 06:03:01G | 02:01:02G | 19:01:01G | A | + | Ext DNA |
| PT0000005 | B529068 | 01:01:01G | — | x | x | 02:01:01G | 05:03:01G | 03:01:01G | 16:01:01 | A | + | Ext DNA |
| PT0000005 | B529069 | x | x | x | x | 05:01:01G | 06:09:01G | 9:01:01 | 26:01:02 | A | + | Ext DNA |
| PT0000005 | B529074 | 01:01:01G | — | x | x | 02:01:01G | 04:02:01G | 01:01:01G | 04:01:01G | B | + | Ext DNA |
| PT0000005 | B529079 | x | x | x | x | 02:01:01G | 05:03:01G | 04:01:01G | 04:02:01G | A | + | Ext DNA |
| PT0000005 | B529081 | x | x | 1:01:01 | — | 02:01:01G | 06:02:01G | 01:01:01G | 04:01:01G | A | + | Ext DNA |
| PT0000005 | B529089 | 01:01:01G | — | x | x | 02:01:01G | 06:09:01G | 10:01 | 11:01:01 | A | + | Ext DNA |
| PT0000005 | B529093 | x | x | x | x | 03:01:01G | 05:01:01G | 03:01:01G | 04:01:01G | B | + | Ext DNA |
| PT0000004 | B530001 | 01:01:01G | — | x | x | 02:01:01G | 05:03:01G | 04:01:01G | — | A | + | Ext DNA |
| PT0000004 | B530002 | 01:01:01G | — | x | x | 03:01:01G | 03:02:01G | 02:01:02G | — | A | + | Ext DNA |
| PT0000004 | B530003 | 01:01:01G | — | x | x | 03:01:01G | 06:03:01G | 02:01:02G | 04:01:01G | A | − | Ext DNA |
| PT0000004 | B530004 | x | x | 1:01:01 | 01:02:01G | 05:01:01G | 06:02:01G | 02:01:02G | 13:01:01G | A | + | Ext DNA |
| PT0000004 | B530005 | 01:01:01G | — | x | x | 03:03:02G | 06:09:01G | 02:01:02G | 04:01:01G | B | + | Ext DNA |
| PT0000004 | B530006 | 01:01:01G | — | x | x | 03:02:01G | 06:03:01G | 04:01:01G | — | AB | + | Ext DNA |
| PT0000004 | B530007 | x | x | 1:01:01 | — | 02:01:01G | 06:02:01G | 02:01:02G | 04:01:01G | A | + | Ext DNA |
| PT0000004 | B530009 | 01:01:01G | — | x | x | 03:02:01G | 05:01:01G | 02:01:02G | 13:01:01G | B | + | Ext DNA |
| PT0000004 | B530010 | 01:01:01G | — | x | x | 02:01:01G | 03:02:01G | 04:01:01G | — | A | + | Ext DNA |
| PT0000004 | B530011 | x | x | x | x | 02:01:01G | — | 01:01:01G | 02:01:02G | A | + | Ext DNA |
| PT0000004 | B530012 | x | x | x | x | 03:01:01G | — | 04:01:01G | 10:01 | O | + | Ext DNA |
| PT0000004 | B530013 | 01:01:01G | — | x | x | 02:01:01G | 03:03:02G | 04:01:01G | 11:01:01 | O | + | Ext DNA |

TABLE 2
Table listing the Super Donor cell lines of the Living Database

| Batch Id | Client Id | A1 | A2 | B1 | B2 | C1 | C2 | DRB11 | DRB12 | DRB31 | DRB32 | DRB41 |
|---|---|---|---|---|---|---|---|---|---|---|---|---|
| PT00000050 | B528022 | 33:03:01G | — | 58:01:01G | — | 03:02:01G | — | 04:04:01 | 13:02:01 | 03:01:01G | — | 01:01:01G |
| PT00000048 | B530011 | 01:01:01G | — | 08:01:01G | — | 07:01:01G | — | 03:01:01G | — | 01:01:02G | — | x |
| PT00000048 | B530015 | 02:01:01G | — | 44:02:01G | — | 05:01:01G | — | 04:01:01 | 13:02:01 | 03:01:01G | — | 01:01:01G |
| PT00000048 | B530017 | 02:01:01G | — | 44:02:01G | — | 05:01:01G | — | 01:01:01G | 04:01:01 | x | x | 01:01:01G |
| PT00000048 | B531063 | 33:03:01G | — | 15:16:01 | — | 14:02:01G | — | 07:01:01G | 15:03:01G | x | x | 01:01:01G |
| PT00000047 | B532080 | 11:01:01G | — | 35:03:01G | — | 04:01:01G | 12:03:01G | 04:03:01 | 07:01:01G | x | x | 01:01:01G |
| PT00000045 | B533003 | 29:02:01G | — | 44:03:01G | — | 16:01:01G | — | 01:01:01G | 07:01:01G | x | x | 01:01:01G |
| PT00000045 | B533040 | 02:01:01G | — | 44:02:01G | — | 05:01:01G | — | 04:01:01 | 13:01:01G | 02:02:01G | — | 01:01:01G |
| PT00000045 | B534078 | 24:02:01G | — | 15:35 | — | 07:02:01G | — | 04:05:01 | 15:02:01 | x | x | 01:01:01G |
| PT00000042 | B536070 | 33:01:01G | — | 14:02:01 | — | 08:02:01G | — | 01:02:01 | — | x | x | x |
| PT00000042 | B536072 | 03:01:01G | — | 35:01:01G | — | 04:01:01G | — | 01:01:01G | 04:03:01 | x | x | 01:01:01G |
| PT00000041 | B537059 | 29:02:01G | — | 44:03:01G | — | 16:01:01G | — | 07:01:01G | 15:01:01G | x | x | 01:01:01G |
| PT00000039 | B538005 | 11:01:01G | — | 44:03:02G | — | 04:01:01G | 05:01:01G | 03:01:01G | 07:01:01G | 02:02:01G | — | 01:01:01G |
| PT00000040 | B538032 | 02:01:01G | — | 07:02:01G | — | 07:02:01G | — | 04:04:01 | 15:01:01G | x | x | 01:01:01G |
| PT00000040 | B538072 | 02:01:01G | — | 07:02:01G | — | 07:02:01G | — | 07:01:01G | 15:01:01G | x | x | 01:01:01G |
| PT00000038 | B539011 | 02:01:01G | — | 15:01:01G | — | 03:03:01G | 12:03:01G | 14:01:01G | 15:01:01G | 02:02:01G | — | x |
| PT00000037 | B540016 | 02:01:01G | — | 15:01:01G | — | 03:03:01G | — | 13:01:01G | — | 01:01:02G | 02:02:01G | x |
| PT00000038 | B540021 | 01:01:01G | — | 08:01:01G | — | 07:01:01G | — | 03:01:01G | — | 01:01:02G | — | x |
| PT00000038 | B540022 | 03:01:01G | — | 35:01:01G | — | 04:01:01G | — | 07:01:01G | 11:02:01 | 02:02:01G | — | 01:01:01G |
| PT00000038 | B540082 | 68:01:02G | — | 40:02:01G | — | 03:04:01G | — | 04:04:01 | 14:02:01 | 01:01:02G | — | 01:01:01G |
| PT00000038 | B540098 | 01:01:01G | — | 37:01:01G | — | 06:02:01G | 06:14 | 04:01:01 | 13:02:01 | 03:01:01G | — | 01:01:01G |
| PT00000037 | B541096 | 30:01:01G | — | 13:02:01G | — | 06:02:01G | — | 07:01:01G | — | x | x | 01:01:01G |
| PT00000036 | B542032 | 02:01:01G | — | 44:02:01G | — | 05:01:01G | — | 04:01:01 | 11:02:01 | 02:02:01G | — | 01:01:01G |
| PT00000036 | B542051 | 02:01:01G | — | 40:01:01G | — | 03:04:01G | — | 01:01:01G | 03:01:01G | 01:01:02G | — | x |
| PT00000035 | B543047 | 03:01:01G | — | 07:02:01G | — | 07:02:01G | — | 10:01:01 | 15:01:01G | x | x | x |
| PT00000035 | B543100 | 02:01:01G | — | 07:02:01G | — | 07:02:01G | — | 09:01:02G | 15:01:01G | x | x | 01:01:01G |
| PT00000032 | B545001 | 01:01:01G | — | 08:01:01G | — | 07:01:01G | — | 03:01:01G | 13:01:01G | 01:01:02G | — | x |
| PT00000032 | B546086 | 24:02:01G | — | 15:01:01G | — | 03:03:01G | — | 04:04:01 | 09:01:02G | x | x | 01:01:01G |
| PT00000032 | B546094 | 02:01:01G | — | 40:01:01G | — | 07:02:01G | — | 08:03:02 | 09:01:02G | x | x | 01:01:01G |
| PT00000030 | B547024 | 01:01:01G | — | 08:01:01G | — | 07:01:01G | — | 03:01:01G | 13:01:01G | 01:01:02G | — | x |
| PT00000029 | B548029 | 02:01:01G | — | 14:01:01 | — | 08:02:01G | — | 07:01:01G | — | x | x | 01:01:01G |
| PT00000030 | B548086 | 11:01:01G | — | 35:01:01G | — | 04:01:01G | — | 01:01:01G | 12:01:01G | 02:02:01G | — | x |
| PT00000028 | B550093 | 01:01:01G | — | 08:01:01G | — | 07:01:01G | — | 03:01:01G | 16:01:01 | 01:01:02G | — | x |
| PT00000026 | B551053 | 02:01:01G | — | 15:01:01G | — | 03:03:01G | 03:04:01G | 04:01:01 | 13:01:01G | 02:02:01G | — | 01:01:01G |
| PT00000026 | B551082 | 26:01:01G | — | 38:01:01 | — | 12:03:01G | — | 04:02:01 | — | x | x | 01:01:01G |
| PT00000025 | B552045 | 02:07:01G | — | 46:01:01G | — | 01:02:01G | 01:03:01G | 09:01:02G | — | x | x | 01:01:01G |
| PT00000025 | B552074 | 03:01:01G | — | 07:02:01G | — | 07:02:01G | — | 11:01:01G | 15:01:01G | 02:02:01G | — | x |
| PT00000026 | B552095 | 24:02:01G | — | 14:02:01 | — | 08:02:01G | — | 01:02:01 | — | x | x | x |
| PT00000024 | B553031 | 02:01:01G | — | 44:02:01G | — | 05:01:01G | 07:04:01G | 04:01:01 | 11:01:01G | 02:02:01G | — | 01:01:01G |
| PT00000023 | B554016 | 01:01:01G | — | 08:01:01G | — | 07:01:01G | — | 03:01:01G | — | 01:01:02G | — | x |
| PT00000023 | B554030 | 31:01:02G | — | 40:01:01G | — | 03:04:01G | — | 04:04:01 | — | x | x | 01:01:01G |
| PT00000023 | B554086 | 02:01:01G | — | 15:01:01G | — | 01:02:01G | 04:01:01G | 01:01:01G | 04:04:01 | x | x | 01:01:01G |
| PT00000021 | B555002 | 02:01:01G | — | 44:02:01G | — | 05:01:01G | — | 04:01:01 | 13:01:01G | 01:01:02G | — | 01:01:01G |
| PT00000021 | B556092 | 02:01:01G | — | 07:02:01G | — | 07:02:01G | — | 04:01:01 | 13:02:01 | 03:01:01G | — | 01:01:01G |
| PT00000020 | B557032 | 24:02:01G | — | 38:02:01G | — | 07:02:01G | — | 04:03:01 | 15:02:01 | x | x | 01:01:01G |
| PT00000020 | B557037 | 01:01:01G | — | 08:01:01G | — | 07:01:01G | — | 03:01:01G | 04:01:01 | 01:01:02G | — | 01:01:01G |
| PT00000018 | B559045 | 02:01:01G | — | 44:02:01G | — | 05:01:01G | — | 04:01:01 | — | x | x | 01:01:01G |
| PT00000018 | B559055 | 01:01:01G | — | 08:01:01G | — | 07:01:01G | — | 03:01:01G | — | 01:01:02G | — | x |
| PT00000017 | B560007 | 29:02:01G | — | 44:03:01G | — | 16:01:01G | — | 07:01:01G | — | x | x | 01:01:01G |
| PT00000016 | B561096 | 01:01:01G | — | 08:01:01G | — | 07:01:01G | — | 01:01:01G | 15:01:01G | 01:01:02G | — | x |
| PT00000015 | B562074 | 01:01:01G | — | 08:01:01G | — | 07:01:01G | — | 03:01:01G | — | 01:01:02G | — | x |
| PT00000013 | B563006 | 01:01:01G | — | 08:01:01G | — | 07:01:01G | — | 01:01:01G | 03:01:01G | 01:01:02G | — | x |
| PT00000014 | B563057 | 02:01:01G | — | 08:01:01G | — | 07:01:01G | — | 03:01:01G | — | 01:01:02G | — | x |
| PT00000014 | B563081 | 01:01:01G | — | 08:01:01G | — | 07:01:01G | — | 03 01:01G | — | 01:01:02G | — | x |

| Batch Id | Client Id | DRB42 | DRB51 | DRB52 | DQB11 | DQB12 | DPB11 | DPB12 | ABO | RHD | Sample Type |
|---|---|---|---|---|---|---|---|---|---|---|---|
| PT00000050 | B528022 | — | x | x | 03:02:01G | 06:09:01G | 05:01:01G | — | O | + | ExtDNA |
| PT00000048 | B530011 | x | x | x | 02:01:01G | — | 01:01:01G | 02:01:02G | A | + | ExtDNA |
| PT00000048 | B530015 | — | x | x | 03:01:01G | 06:04:01G | 04:01:01G | 17:01:01G | O | − | ExtDNA |
| PT00000048 | B530017 | — | x | x | 03:01:01G | 05:01:01G | 04:01:01G | 04:02:01G | A | + | ExtDNA |
| PT00000048 | B531063 | — | 01:01:01 | — | 02:01:01G | 06:02:01G | 01:01:01G | 17:01:01G | O | + | ExtDNA |
| PT00000047 | B532080 | — | x | x | 03:02:01G | 03:03:02G | 02:01:02G | 04:01:01G | B | + | ExtDNA |
| PT00000045 | B533003 | — | x | x | 02:01:01G | 05:01:01G | 11:01:01 | — | B | + | ExtDNA |
| PT00000045 | B533040 | — | x | x | 03:01:01G | 06:03:01G | 02:01:02G | 04:01:01G | O | − | ExtDNA |
| PT00000045 | B534078 | — | 01:01:01 | — | 04:01:01G | 05:02:01G | 01:01:01G | — | O | + | ExtDNA |
| PT00000042 | B536070 | x | x | x | 05:01:01G | — | 03:01:01G | 04:02:01G | O | + | ExtDNA |
| PT00000042 | B536072 | — | x | x | 03:02:01G | 05:01:01G | 02:01:02G | 04:02:01G | A | + | ExtDNA |
| PT00000041 | B537059 | — | 01:01:01 | — | 02:01:01G | 06:03:01G | 03:01:01G | 11:01:01 | O | − | ExtDNA |
| PT00000039 | B538005 | — | x | x | 02:01:01G | — | 02:01:02G | 04:01:01G | A | + | Misc |
| PT00000040 | B538032 | — | 01:01:01 | — | 03:02:01G | 06:02:01G | 03:01:01G | 04:01:01G | O | + | Misc |
| PT00000040 | B538072 | — | 01:01:01 | — | 02:01:01G | 06:02:01G | 01:01:01G | 04:01:01G | B | + | Misc |
| PT00000038 | B539011 | x | 01:01:01 | — | 05:03:01G | 06:03:01G | 04:01:01G | — | A | + | Misc |
| PT00000037 | B540016 | x | x | x | 06:02:01G | 06:03:01G | 03:01:01G | 04:01:01G | O | + | Misc |
| PT00000038 | B540021 | x | x | x | 02:01:01G | — | 01:01:01G | 10:01 | O | + | Misc |
| PT00000038 | B540022 | — | x | x | 02:01:01G | 03:01:01G | 01:01:01G | 03:01:01G | O | + | Misc |
| PT00000038 | B540082 | — | x | x | 03:01:01G | 03:02:01G | 04:01:01G | 04:02:01G | O | + | Misc |
| PT00000038 | B540098 | — | x | x | 03:02:01G | 06:04:01G | 02:01:02G | 13:01:01G | O | + | Misc |
| PT00000037 | B541096 | — | x | x | 02:01:01G | — | 09:01:01 | 17:01:01G | A | + | Misc |
| PT00000036 | B542032 | — | x | x | 03:01:01G | — | 04:01:01G | — | A | + | Misc |
| PT00000036 | B542051 | x | x | x | 02:01:01G | 05:01:01G | 01:01:01G | 04:02:01G | O | + | Misc |
| PT00000035 | B543047 | x | 01:01:01 | — | 05:01:01G | 06:02:01G | 02:01:02G | 04:01:01G | A | + | Misc |
| PT00000035 | B543100 | — | 01:01:01 | — | 03:03:02G | 06:02:01G | 04:01:01G | 04:02:01G | O | + | Misc |
| PT00000032 | B545001 | x | x | x | 02:01:01G | 06:03:01G | 01:01:01G | 02:01:02G | B | + | Misc |
| PT00000032 | B546086 | — | x | x | 03:02:01G | 03:03:02G | 04:01:01G | — | A | + | Misc |
| PT00000032 | B546094 | — | x | x | 03:03:02G | 06:01:01G | 05:01:01G | 36:01 | A | + | Misc |
| PT00000030 | B547024 | x | x | x | 02:01:01G | 06:03:01G | 01:01:01G | 23:01:01G | O | + | Misc |
| PT00000029 | B548029 | — | x | x | 02:01:01G | — | 04:01:01G | 04:02:01G | AB | + | Misc |
| PT00000030 | B548086 | x | x | x | 03:01:01G | 05:01:01G | 02:01:02G | 06:01 | A | + | Misc |
| PT00000028 | B550093 | x | 02:02:01G | — | 02:01:01G | 05:02:01G | 04:01:01G | — | B | + | Misc |
| PT00000026 | B551053 | — | x | x | 03:02:01G | 06:03:01G | 01:01:01G | 04:01:01G | A | + | Misc |
| PT00000026 | B551082 | — | x | x | 03:02:01G | — | 04:01:01G | 04:02:01G | A | + | Misc |
| PT00000025 | B552045 | — | x | x | 03:03:02G | — | 05:01:01G | — | A | + | Misc |
| PT00000025 | B552074 | x | 01:01:01 | — | 03:01:01G | 06:02:01G | 04:01:01G | 20:01:01 | A | + | Misc |
| PT00000026 | B552095 | x | x | x | 05:01:01G | — | 04:01:01G | — | O | + | Misc |
| PT00000024 | B553031 | — | x | x | 03:01:01G | — | 04:01:01G | — | O | + | Misc |
| PT00000023 | B554016 | x | x | x | 02:01:01G | — | 04:01:01G | — | A | + | Misc |
| PT00000023 | B554030 | — | x | x | 03:02:01G | — | 02:01:02G | 11:01:01 | B | + | Misc |
| PT00000023 | B554086 | — | x | x | 03:02:01G | 05:01:01G | 04:01:01G | 10:01 | O | + | Misc |
| PT00000021 | B555002 | — | x | x | 03:02:01G | 06:03:01G | 02:01:02G | 04:01:01G | A | + | Misc |
| PT00000021 | B556092 | — | x | x | 03:01:01G | 06:04:01G | 03:01:01G | 04:01:01G | B | + | Misc |
| PT00000020 | B557032 | — | 01:01:01 | — | 03:02:01G | 05:02:01G | 01:01:01G | 05:01:01G | O | + | Misc |
| PT00000020 | B557037 | — | x | x | 02:01:01G | 03:02:01G | 04:01:01G | 04:02:01G | A | + | Misc |
| PT00000018 | B559045 | — | x | x | 03:01:01G | — | 04:01:01G | — | O | + | Misc |
| PT00000018 | B559055 | x | x | x | 02:01:01G | — | 01:01:01G | 04:01:01G | A | + | Misc |
| PT00000017 | B560007 | — | x | x | 02:01:01G | — | 11:01:01 | — | A | − | Misc |
| PT00000016 | B561096 | x | 01:01:01 | — | 02:01:01G | 06:02:01G | 04:01:01G | 04:02:01G | A | − | Misc |
| PT00000015 | B562074 | x | x | x | 02:01:01G | — | 01:01:01G | 20:01:01 | O | + | Misc |
| PT00000013 | B563006 | x | x | x | 02:01:01G | 05:01:01G | 01:01:01G | 04:01:01G | O | + | Misc |
| PT00000014 | B563057 | x | x | x | 02:01:01G | — | 01:01:01G | 02:01:02G | A | + | Misc |
| PT00000014 | B563081 | x | x | x | 02:01:01G | — | 01:01:01G | 17:01:01G | O | − | Misc |

### Example 2: Constructive Example Showing Population HLA Matching

The present Constructive Example demonstrates HLA haplotypes can be matched to multiple populations. Such multi-population matched HLA types can be stored in an HLA-indexed cell repository and living database, as discussed above.

Overlap of HLA types between American people and Japanese people was analyzed using publicly available HLA datasets. For American people, HLA type data was used from BE THE MATCH websites (Human Immunology (2007) 68, 779-788). For Japanese people, HLA type was used from the HLA laboratory website). From a sample size of 27,996 American people, it was determined that there were 6,779 HLA types. Similarly, from a sample size of 8,138 Japanese people, it was determined that there were 2,796 HLA types. It was also determined that the above two datasets had 751 overlapping HLA types, indicating a significant overlap in HLA types between American and Japanese people. Of the 6,779 HLA types for American people, 152 HLA types were found to be representative of 90% of the population. These 152 HLA types that were found to be more common in the American people have also been matched.

An HLA-indexed database of cells and/or cell lines and/or tissue derived therefrom may advantageously identify the highest-ranking homozygous loci (e.g., HLA-A, -B, and -DRB1) combinations for a given population (e.g., the U.S., Europe, Japan, China, etc.). In this way, one or more cell lines each corresponding to a super donor (or other compatible individual cell line) for a given subject in need of a transplant, biological tissue, and/or biological fluid, for example, may be identified very quickly for the subject, thereby significantly reducing wait times for matched organ donors.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of querying and retrieving data entries of a HLA-indexed database that match a set of queried human leukocyte antigen (HLA) loci for identification, production, and/or retrieval of hematopoietic stem cells (HSCs) suitable for treatment of a subject, said method comprising the steps of:
storing, by a processor of a computing device, the database comprising a data entry corresponding to each of a plurality of characterized induced pluripotent stem cell (iPSC) lines, the data entry for each of the plurality of characterized iPSC lines comprising a set of characterized HLA loci corresponding to the iPSC line;
receiving, by the processor, a query from a user, the query comprising the set of queried HLA loci for the subject; and
retrieving, by the processor, one or more data entries of the database, each representative of an iPSC line matching the set of queried HLA loci.

2. The method of claim 1, wherein each of the one or more retrieved data entries of the database is representative of an iPSC cell line in a repository and/or a cell line in the repository corresponding to an iPSC cell line.

3. The method of claim 1 or claim 2, wherein each of the iPSC lines corresponding to the one or more data entries of the database is stored in a physical repository.

4. The method of claim any one of the preceding claims, wherein the set of characterized HLA loci corresponding to each of the plurality of characterized iPSC lines comprises each of a set of at least 3 given loci, wherein the at least 3 given loci are HLA-A, HLA-B, and HLA-DRB.

5. The method of any one of the preceding claims, wherein the set of characterized HLA loci corresponding to each of the plurality of characterized iPSC lines comprises at least 9 given loci, wherein the at least 9 given loci are HLA-A, HLA-B, HLA-C, HLA-DRB1, HLA-DRB3, HLA-DRB4, HLA-DRB5, HLA-DQB1, and HLA-DPB1.

6. The method of any one of the preceding claims, wherein the set of characterized HLA loci corresponding to each of the plurality of characterized iPSC lines comprises at least 3 given loci selected from the group consisting of HLA-A, HLA-B, HLA-C, HLA-DRB1, HLA-DRB3, HLA-DRB4, HLA-DRB5, HLA-DQB1, and HLA-DPB1.

7. The method of any one of the preceding claims, wherein each of the one or more retrieved data entries of the database exactly match or partially match the set of queried HLA loci for the subject.

8. The method of any one of the preceding claims, further comprising retrieving one or more cells from the physical repository corresponding to the one or more retrieved data entries.

9. The method of any one of the preceding claims, wherein the data entry corresponding to each of a plurality of characterized induced pluripotent stem cell (iPSC) lines further comprises ABO blood type and the query further comprises ABO blood type, and wherein the retrieving step comprises retrieving, by the processor, one or more data entries, each representative of the iPSC line matching the set of queried HLA loci and the queried ABO blood type.

10. The method of any one of the preceding claims, wherein the data entry corresponding to each of a plurality of characterized induced pluripotent stem cell (iPSC) lines further comprises RHD blood group and the query further comprises RHD blood group, and wherein the retrieving step comprises retrieving, by the processor, one or more data entries, each representative of the iPSC line matching the queried RHD blood group and the set of queried HLA loci.

11. The method of any one of the preceding claims, wherein the set of queried HLA loci correspond to the subject in need of an HLA match.

12. The method of claim 11, wherein the HLA match is each of the iPSC lines corresponding to each of the one or more retrieved data entries of the database that exactly match or partially match the set of queried HLA loci of the subject.

13. The method of any one of the preceding claims, wherein the set of queried HLA loci is determined by processing and analyzing a biological sample from the subject in need of an HLA match.

14. The method of any one of the preceding claims, further comprising retrieving characterized cells from the physical repository, wherein the characterized cells correspond to the one or more retrieved data entries matching the set of queried HLA loci.

15. The method of any one of the preceding claims, further comprising producing blood progenitors and/or HSCs from an iPSC line corresponding to the one or more retrieved data entries matching the set of queried HLA loci.

16. The method of any one of the preceding claims, further comprising administering blood progenitors and/or HSCs to the subject, wherein said blood progenitors and/or HSCs are derived from an iPSC line corresponding to the one or more retrieved data entries matching the set of queried HLA loci.

17. The method of claim 16, wherein the administering step comprises administering the blood progenitors and/or the HSCs to the subject for treatment of a known disease or condition in the subject, wherein the known disease or condition is a member selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

18. The method of any one of the preceding claims, wherein the database comprises a data entry corresponding to each of a plurality of iPSC super donor cell lines, wherein the data entry for each super donor cell line comprises a set of characterized HLA loci corresponding to the super donor cell line.

19. The method of claim 18, wherein each of the plurality of the iPSC super donor cell lines are used for treatment of the subject with lower risk of immune rejection by the subject.

20. The method of claim 18 or claim 19, further comprising determining the set of characterized HLA loci corresponding to each of the plurality of the super donor cell lines by processing and analyzing one or more biological samples collected from each of one or more super donor individuals.

21. The method of claim 20, wherein the step of determining the set of characterized HLA loci corresponding to each of the plurality of the super donor cell lines comprises identifying a set of at least 3 HLA loci, wherein the at least 3 HLA loci are HLA-A, HLA-B, and HLA-DRB.

22. The method of claim 20, wherein the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1.

23. The method of claim 22, wherein the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

24. The method of any one of claims 18 to 23, wherein the set of characterized HLA loci corresponding to each of the plurality of the super donor cell lines comprises at least 3 homozygous HLA loci selected from the group consisting of HLA-A, HLA-B, and DRB.

25. The method of claim 24, wherein the homozygous set of characterized HLA loci belong to a set of most-common HLA loci for a given population that matches a majority of the given population.

26. The method of any one of claims 18 to 25, wherein the homozygous set of characterized HLA loci comprise homozygous HLA loci in at least 3 major sites, wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

27. The method of any one of claims 18 to 26, wherein the plurality of iPSC super donor cell lines match at least 70% of the population from which the subject originates.

28. A system of querying and retrieving data entries of an HLA-indexed database that match a set of queried human leukocyte antigen (HLA) loci for identification, production, and/or retrieval of hematopoietic stem cells (HSCs) suitable for treatment of a subject, the system comprising:
a physical repository comprising a plurality of cells corresponding to characterized induced pluripotent stem cell (iPSC) lines;
a processor;
a non-transitory computer readable medium having instructions stored thereon, wherein the instructions, when executed by the processor, cause the processor to:
store the database, said database comprising a data entry corresponding to each of the plurality of characterized iPSC lines in the physical repository, the data entry for each of the plurality of characterized iPSC lines comprising a set of characterized HLA loci corresponding to the iPSC line;
receive a query from a user, the query comprising the set of queried HLA loci for the subject; and
retrieve, by the processor, one or more data entries of the database, each representative of an iPSC line matching the set of queried HLA loci.

29. The system of claim 28, wherein each of the one or more retrieved data entries of the database is representative of an iPSC cell line in the physical repository and/or a cell line in the physical repository corresponding to an iPSC cell line.

30. The system of claim 28 or claim 29, wherein the set of characterized HLA loci corresponding to each of the plurality of characterized iPSC lines comprises each of a set of at least 3 given loci, wherein the at least 3 given loci are HLA-A, HLA-B, and HLA-DRB.

31. The system of claim 28 or claim 29, wherein the set of characterized HLA loci corresponding to each of the plurality of characterized iPSC lines comprises at least 9 given loci, wherein the at least 9 given loci are HLA-A, HLA-B, HLA-C, HLA-DRB1, HLA-DRB3, HLA-DRB4, HLA-DRB5, HLA-DQB1, and HLA-DPB1.

32. The system of claim 31, wherein the set of characterized HLA loci corresponding to each of the plurality of characterized iPSC lines comprises at least 3 given loci selected from the group consisting of HLA-A, HLA-B, HLA-C, HLA-DRB1, HLA-DRB3, HLA-DRB4, HLA-DRB5, HLA-DQB1, and HLA-DPB1.

33. The system of any one of claims 28 to 32, wherein each of the one or more retrieved data entries of the database exactly match or partially match the set of queried HLA loci for the subject.

34. The system of any one of claims 28 to 33, wherein the data entry corresponding to each of the plurality of characterized induced pluripotent stem cell (iPSC) lines further comprises ABO blood type and the query further comprises ABO blood type, and wherein the processor retrieves one or more data entries, each representative of the iPSC line matching the set of queried HLA loci and the queried ABO blood type.

35. The system of any one of claims 28 to 34, wherein the data entry corresponding to each of the plurality of characterized induced pluripotent stem cell (iPSC) lines further comprises RHD blood group and the query further comprises RHD blood group, and wherein the processor retrieves one or more data entries, each representative of the iPSC line matching the queried RHD blood group and the set of queried HLA loci.

36. The system of any one of claims 28 to 35, wherein the set of queried HLA loci correspond to the subject in need of an HLA match.

37. The system of claim 36, wherein the HLA match is each of the iPSC lines corresponding to each of the one or more retrieved data entries of the database that exactly match or partially match the set of queried HLA loci of the subject.

38. The system of any one of claims 28 to 37, wherein the physical repository comprises one or more liquid nitrogen storage tanks.

39. The system of any one of claims 28 to 38, wherein the database comprises a data entry corresponding to each of a plurality of iPSC super donor cell lines, wherein the data entry for each super donor cell line comprises a set of characterized HLA loci corresponding to the super donor cell line.

40. The system of claim 39, wherein each of the plurality of the iPSC super donor cell lines can be used for treatment of the subject with lower risk of immune rejection by the subject.

41. The system of any one of claims 30 to 40, wherein the set of characterized HLA loci corresponding to each of the plurality of the super donor cell lines comprises a set of at least 3 HLA loci, wherein the at least 3 HLA loci are HLA-A, HLA-B, and HLA-DRB.

42. The system of claim 41, wherein the set of characterized HLA loci corresponding to each of the plurality of the super donor cell lines comprises a set of at least 9 HLA loci, wherein the at least 9 HLA loci are HLA-A, HLA-B, HLA-C, HLA-DRB1, HLA-DRB3, HLA-DRB4, HLA-DRB 5, HLA-DQB1, and HLA-DPB1.

43. The system of claim 42, wherein the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

44. The system of any one of claims 39 to 43, wherein the set of characterized HLA loci corresponding to each of the plurality of the super donor cell lines comprises at least 3 homozygous HLA loci selected from the group consisting of HLA-A, HLA-B, and DRB.

45. The system of claim 44, wherein the homozygous set of characterized HLA loci belong to a set of most-common HLA loci for a given population that matches a majority of the given population

46. The system of any one of claims 39 to 45, wherein the homozygous set of characterized HLA loci comprise homozygous HLA loci in at least 3 major sites, wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

47. The system of any one of claims 39 to 46, wherein the plurality of iPSC super donor cell lines match at least 70% of the population from which the subject originates.

48. The system of any one of claims 28 to 47, wherein the plurality of cells corresponding to characterized iPSC lines in the physical repository are capable of being cultured, expanded, stored, partially and/or fully differentiated, and transferred to a subject.

49. The system of any one of claims 28 to 48, wherein the physical repository is a biorepository for collecting, processing, storing, and/or distributing biospecimens, wherein each of the biospecimens is a member selected from the group consisting of iPSCs, iPSC-derived cells, and tissue created from iPSCs.

50. The system of any one of claims 28 to 49, wherein the physical repository is in communication with one or more processors programmed for identifying, locating, and/or inventorying the biospecimens in the physical repository.

51. The system of any one of claims 28 to 50, wherein the physical repository is outfitted with hardware and/or robotics for automated sample handling.

52. The system of any one of claims 28 to 51, comprising characterized super donor cells.

53. Use of the system of any one of claims 28 to 52 for one or more clinical procedures.

54. The use of the system of claim 53, wherein each of the one or more clinical procedures is a member selected from the group consisting of gene therapy, cell transplant, and tissue transplant.

55. Use of the system of any one of claims 28 to 52 for one or more pre-clinical studies.

56. The use of the system of claim 55, wherein each of the one or more pre-clinical studies is a member selected from the group consisting of in vitro screens, in vivo screens, efficacy testing of medications, toxicity testing of medications, and testing for use in personalized medicine.

57. A method of treating a subject, the method comprising:
administering blood progenitors and/or HSCs to the subject, wherein said blood progenitors and/or HSCs are produced from an iPSC line corresponding to the one or more retrieved data entries matching a set of queried HLA loci, wherein iPS cells from the iPSC line are stored in and retrieved from the physical repository of any one of system claims 28 to 52.
